SEQUENCE LISTING (9 sequences)

SEQ ID NO: 1 - protein, 5 aa, Artificial Sequence
  synthetic mature cholix toxin (CT) and cholera exotoxin (CET) carboxy-terminal sequence
KDELK

SEQ ID NO: 2 - protein, 5 aa, Artificial Sequence
  synthetic Pseudomonas endotoxin (PE) carboxy-terminal native sequence, residues 609-613
REDLK

SEQ ID NO: 3 - protein, 4 aa, Artificial Sequence
  synthetic modified carboxy-terminal cytosol translocation sequence in Pseudomonas endotoxin (PE)
REDL

SEQ ID NO: 4 - protein, 4 aa, Artificial Sequence
  synthetic modified carboxy-terminal cytosol translocation sequence in Pseudomonas endotoxin (PE)
KDEL

SEQ ID NO: 5 - protein, 8 aa, Artificial Sequence
  synthetic mature cholix toxin (CT) eight residue leader sequence 6-histidine tag flanked by methionine on each side
MHHHHHHM

SEQ ID NO: 6 - protein, 9 aa, Artificial Sequence
  synthetic mature cholix toxin (CT) and cholera exotoxin (CET) NAD domain
GGEDETVIG

SEQ ID NO: 7 - protein, 9 aa, Artificial Sequence
  synthetic Pseudomonas endotoxin (PE) NAD binding site
GGRLETILG

SEQ ID NO: 8 - RNA, 12 nt, Artificial Sequence
  synthetic sarcin-ricin loop conserved eukaryotic ribosome sequence
aguacgagagga

SEQ ID NO: 9 - protein, 4 aa, Artificial Sequence
  synthetic caspase 3 substrate
DEVD

(19) United States
(12) Patent Application Publication
(10) Pub. No.: US 2012/0276190 A1
(43) Pub. Date: Nov. 1, 2012

(54) THERAPY WITH A CHIMERIC MOLECULE AND A PRO-APOPTOTIC AGENT

(75) Inventor: David J. Fitzgerald, Rockville, MD (US)

(73) Assignee: The Government of the U.S.A. as Represented By the Secretary of the Dept. of Health & Human Services, Rockville, MD (US)

(21) Appl. No.: 13/406,480

(22) Filed: Feb. 27, 2012

Related U.S. Application Data
(63) Continuation-in-part of application No. PCT/US10/46382, filed on Aug. 23, 2010.
(60) Provisional application No. 61/238,032, filed on Aug. 28, 2009.

(51) Int. Cl. A61K 39/395; C12N 5/09; A61P 35/00; A61K 9/127

(52) U.S. Cl. 424/450; 424/183.1; 435/375

(57) ABSTRACT

The present invention provides compositions comprising a chimeric molecule comprising a cytotoxin that inhibits protein synthesis and an agent that inactivates an anti-apoptotic BCL-2 family member protein and methods of inhibiting the growth of or promoting the apoptosis of an aberrantly proliferating cell population by co-administering the chimeric molecule and the agent that inactivates an anti-apoptotic BCL-2 family member protein.

### CROSS-REFERENCES TO RELATED APPLICATIONS

This application is a continuation-in-part of International Application Serial No. PCT/US2010/046382, filed on Aug. 23, 2010 and designating the United States, which claimed priority benefit of U.S. Provisional Application Ser. No. 61/238,032, filed on Aug. 28, 2009, the disclosures of each of which are incorporated herein by reference for all purposes.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED AS AN ASCII TEXT FILE

The Sequence Listing written in file-585-1-1.TXT, created on Feb. 27, 2012, 4,096 bytes, machine format IBM-PC, MS-Windows operating system, is hereby incorporated by reference in its entirety for all purposes.

### FIELD OF THE INVENTION

The present invention provides compositions comprising a chimeric molecule comprising a cytotoxin that inhibits protein synthesis and an agent that inactivates an anti-apoptotic BCL-2 family member protein and methods of inhibiting the growth of or promoting the apoptosis of an aberrantly proliferating cell by co-administering the chimeric molecule and the agent that inactivates an anti-apoptotic BCL-2 family member protein.

### BACKGROUND OF THE INVENTION

Antibody-based therapies of human cancer have become first line treatments in certain settings. By way of example, Her2-positive breast cancer patients are treated with Herceptin (Hudis, 2007, N Engl J Med 357:39-51) while individuals with certain B-cell malignancies receive Rituxan (Cheson and Leonard, 2008, N Engl J Med 359:613-26). These antibodies are given either alone or in combination with chemotherapy. The potential benefit of using antibody-based therapy is an effective treatment with low side effects. However, when the administration of an unmodified antibody is not effective, several options are available to make the antibody a ‘cytotoxic’ agent (Heimann and Weiner, 2007, Surg Oncol Clin N Am 16:775-92, viii). Radionuclides, small molecular weight drugs (including prodrugs), enzymes, homing partners (such as bispecific antibodies) and protein toxins have each been “attached” to tumor-binding antibodies as adjuncts to increase their effectiveness (Green et al., 2007, Clin Cancer Res 13:5598s-603s; Rybak, 2008, Curr Pharm Biotechnol 9:226-30; Liu et al., 2008, Immunological Reviews 222:9-27; Singh et al., 2008, Curr Med Chem 15:1802-26; Brumlik et al., 2008, Expert Opin Drug Deliv 5:87-103; Carter and Senter, 2008, Cancer J 14:154-69; Goldenberg and Sharkey, 2007, Oncogene 26:3734-44; Pastan et al., 2007, Annu Rev Med 58:221-37; Kreitman and Pastan, 2006, Hematol Oncol Clin North Am 20:1137-51, viii). Each type of modified antibody has benefits and limitations (Heimann and Weiner, 2007, Surg Oncol Clin N Am 16:775-92, viii; Ricart and Tolcher, 2007, Nat Clin Oncol 4:245-55).

In the past several years immunoconjugates have been developed as an alternative therapeutic approach to treat malignancies. Immunoconjugates were originally composed of an antibody chemically conjugated to a plant or a bacterial protein toxin, a form that is known as an immunotoxin. The antibody binds to the antigen expressed on the target cell and the toxin is internalized, arresting protein synthesis and inducing cell death (Brinkmann, U., Mol. Med. Today, 2:439-446 (1996)). More recently, genes encoding the antibody and the toxin have been fused and the immunotoxin expressed as a fusion protein.

Immunotoxins inhibit protein synthesis but do not always kill the targeted cells. Apparently, some cancer cells resist killing by immunotoxins in the same way they resist chemotherapy.

Recently, inhibitors of anti-apoptotic proteins in the Bcl-2 family have been developed. Compounds of particular interest include agents that mimic the Bcl-2 homology 3 (BH3) domains of the proapoptotic Bcl-2 family members.

There remains a need to improve the efficacy of targeted therapies against aberrantly proliferating cell populations, including cancer cells. The present invention is based, in part, on the unexpected discovery of the co-operative action between cytotoxins that inhibit protein synthesis and inhibitors of anti-apoptotic members of the Bcl-2 family of proteins.

### BRIEF SUMMARY OF THE INVENTION

The present invention provides compositions and methods for co-administering a cytotoxin that inhibits protein synthesis and a pro-apoptotic agent, for use in promoting apoptosis and inhibiting proliferation of aberrantly proliferating cells. Accordingly, in one aspect, the invention provides compositions comprising a chimeric molecule and a pro-apoptotic agent, wherein the chimeric molecule comprises a toxin that inhibits protein synthesis, wherein the pro-apoptotic agent inactivates an anti-apoptotic Bcl-2 family member protein.

In a further aspect, the invention provides methods for promoting apoptosis, inhibiting or reducing proliferation, preventing metastasis, and/or killing a cell, e.g., a cancer cell, comprising contacting the cell with a chimeric molecule and a pro-apoptotic agent, wherein the chimeric molecule comprises a toxin that inhibits protein synthesis, wherein the pro-apoptotic agent inactivates an anti-apoptotic Bcl-2 family member protein.

In a related aspect, the invention provides methods for improving the efficacy of a cytotoxin or an immunotoxin in killing a target cell population, comprising contacting the cell population with a chimeric molecule and a pro-apoptotic agent, wherein the chimeric molecule comprises a toxin that inhibits protein synthesis, wherein the pro-apoptotic agent inactivates an anti-apoptotic Bcl-2 family member protein. Co-administering the chimeric molecule comprising a toxin that inhibits protein synthesis with the pro-apoptotic agent increases the killing of the target cell population, e.g., in comparison to the killing of the target cell population with either the chimeric molecule or the pro-apoptotic agent alone. In some embodiments, the improved killing is at least about 5-fold, 10-fold, 20-fold, 50-fold or 100 fold in comparison to the killing of the target cell population with either the chimeric molecule or the pro-apoptotic agent alone.

In a related aspect, the invention provides methods for enhancing the delivery of a cytotoxin or an immunotoxin to the cytosol of a target cell, comprising contacting the cell with a chimeric molecule and a pro-apoptotic agent, wherein the chimeric molecule comprises cytotoxin, wherein the pro-apoptotic agent inactivates an anti-apoptotic Bcl-2 family member protein. Co-administering the chimeric molecule comprising a cytotoxin with the pro-apoptotic agent increases the delivery to the cytosol of the target cell, e.g., in comparison to the delivery to the cytosol of the target cell of the cytotoxin alone. For the improved delivery methods, the cytotoxin can be, but need not be, enzymatically active or an agent which enters the cytosol from via the endoplasmic reticulum. In some embodiments, the cytotoxin is a protein which has an endoplasmic reticulum retention sequence (e.g., KDEL (SEQ ID NO:4), REDLK (SEQ ID NO:2), or REDL (SEQ ID NO:3)). In some embodiments, the cytotoxin is a protein synthesis inhibitor, or possesses ADP ribosylation activity.

In some embodiments, the chimeric molecule is an immunotoxin comprising an antibody against a cell surface antigen on a tumor cell and a toxin that inhibits protein synthesis and may also further comprise an endoplasmic reticulum retention sequence.

In some embodiments, the cell surface antigen is on a lymphocytic cell, for example, a hematologic cancer cell. In some embodiments, the cell surface antigen is on a cell that overexpresses mesothelin. In some embodiments, the cell surface antigen is selected from the group consisting of CD19, CD21, CD22, CD25, CD30, CD33, CD79b, transferrin receptor, EGF receptor, mesothelin, cadherin and Lewis Y.

In some embodiments, the toxin is an ADP-ribosylating agent. In some embodiments, the toxin is a ribosomal inactivating agent. In some embodiments, the toxin is selected from Pseudomonas exotoxin A, diphtheria toxin, cholix toxin, cholera exotoxin, shiga toxin, ricin toxin and pokeweed antiviral protein (PAP).

In some embodiments, the toxin is a Pseudomonas exotoxin A. In some embodiments, the Pseudomonas exotoxin A is selected from the group consisting of PE25, PE35, PE38, PE40, Domain III of PE, and variants thereof.

In some embodiments, the pro-apoptotic agent inhibits the activity of an anti-apoptotic BCL-2 family member protein. In some embodiments, the pro-apoptotic agent is a BH3-only mimetic. In some embodiments, the pro-apoptotic agent selected from the group consisting of ABT-737, ABT-263, oblimersen sodium, AT-101 and GX15-070. In some embodiments, the pro-apoptotic agent is selected from ABT-263 and ABT-737.

In some embodiments, the antibody is selected from the group consisting of B3, RFB4, SS1, MN and HB21. In some embodiments, the immunotoxin is selected from the group consisting of LMB-2, LMB-7, LMB-9, BL22, HA22, HA22-LR, SS1P and SS1P-LR.

In some embodiments, the pro-apoptotic agent is encapsulated in a liposome that is attached to the immunotoxin.

In some embodiments, the combined agents are contacted with a tumor cell that is a leukemia cell or a lymphoma cell. In some embodiments, the combined agents are contacted with a cancer cell derived from epithelial tissue, e.g., an epithelial cancer, e.g., a carcinoma. In some embodiments, the combined agents are contacted with a tumor cell that overexpresses mesothelin.

In some embodiments, the immunotoxin and the pro-apoptotic agent are administered together. In some embodiments, the immunotoxin and the pro-apoptotic agent are administered separately.

In some embodiments, the immunotoxin and the pro-apoptotic agent are administered concurrently. In some embodiments, the immunotoxin and the pro-apoptotic agent are administered sequentially.

Further embodiments of the invention are described herein.

### DEFINITIONS

Units, prefixes, and symbols are denoted in their Système International de Unites (SI) accepted form. Numeric ranges are inclusive of the numbers defining the range. Unless otherwise indicated, nucleic acids are written left to right in 5′ to 3′ orientation; amino acid sequences are written left to right in amino to carboxy orientation. The headings provided herein are not limitations of the various aspects or embodiments of the invention, which can be had by reference to the specification as a whole. Accordingly, the terms defined immediately below are more fully defined by reference to the specification in its entirety.

A “BCL-2 family member” refers to a family of mammalian genes and the proteins they produce. They govern mitochondrial outer membrane permeabilization (MOMP) and can be either pro-apoptotic (e.g., Bax, Bak, Diva, Noxa, Puma, Bcl-Xs, Bik, Bim, Bad, Bid and Egl-1) or anti-apoptotic (e.g., Bcl-2, Bcl-xL, Bcl-w, Mcl-1, CED-9, Bfl-1/A-1by). There are at least 25 genes in the Bcl-2 family. Structurally, the members of the Bcl-2 family share one or more of the four characteristic domains of homology entitled the Bcl-2 homology (BH) domains (named BH1, BH2, BH3 and BH4). The anti-apoptotic Bcl-2 proteins, such as Bcl-2 and Bcl-xL, conserve all four BH domains. The BH domains also serve to subdivide the pro-apoptotic Bcl-2 proteins into those with several BH domains (e.g. Bax and Bak) or those proteins that have only the BH3 domain (e.g. Bid, Bim and Bad). The Bcl-2 family has a general structure comprising a hydrophobic helix surrounded by amphipathic helices. Many members of the family have transmembrane domains. The site of action for the Bcl-2 family is mostly on the outer mitochondrial membrane. Functionally, The BH3-only pro-apoptotic proteins act upstream in response to a variety of cellular stimuli to propagate the apoptotic signal and induce the activation of Bax and Bak. Antiapoptotic proteins, including Bcl-2, Bcl-xL, Bcl-w, Mcl-1, and Bfl-1, promote cell survival by binding to and sequestering their proapoptotic counterparts, thus preventing Bax/Bak activation. BCL-2 family member proteins are well characterized and reviewed, e.g., in Yip and Reed, Oncogene (2008) 27(50):6398-406; Levine, et al, Autophagy. (2008) 4(5):600-6; and Szegezdi, et al., Am J Physiol Cell Physiol. 2009 May; 296(5):C941-53.

A “BH3-only mimetic” or “BH3-mimetic” interchangeably refer to a class of agents that agents that mimic the Bcl-2 homology 3 (BH3) domains of the proapoptotic Bcl-2 family members. BH3-only mimetics are potent inhibitors of antiapoptotic Bcl-2 family members. Exemplary BH3-only mimetics include ABT-263 and ABT-737. Inhibitors of anti-apoptotic BCL2 family member proteins and BH3-only mimetics are reviewed, e.g., in Kang and Reynolds, Clin Cancer Res (2009) 15(4):1126-32; Azmi and Mohammad, J Cell Physiol (2009) 218:13-21; Lessene, et al., Nat Rev Drug Discov (2008) 7(12):989-1000; Vogler, et al., Cell Death Differ (2009) 16(3):360-7; Labi, et al, Cell Death Differ (2008) 15(6):977-87 and Zhang, et al., Drug Resist Updat. (2007) 10(6):207-17.

“CD22” refers to a lineage-restricted B cell antigen belonging to the Ig superfamily. It is expressed in 60-70% of B cell lymphomas and leukemias and is not present on the cell surface in early stages of B cell development or on stem cells. See, e.g. Vaickus et al., Crit. Rev. Oncol/Hematol. 11:267-297 (1991).

As used herein, the term “anti-CD22” in reference to an antibody that specifically binds CD22 and includes reference to an antibody which is generated against CD22. In preferred embodiments, the CD22 is a primate CD22, such as human CD22. In one preferred embodiment, the antibody is generated against human CD22 synthesized by a non-primate mammal after introduction into the animal of cDNA which encodes human CD22.

“CD25” or “Tac” refers to the alpha chain of the IL-2 receptor (IL2R). It is a type I transmembrane protein present on activated T cells, activated B cells, some thymocytes, myeloid precursors, and oligodendrocytes that associates with CD122 to form a heterodimer that can act as a high-affinity receptor for IL-2. CD25 expressed in most B-cell neoplasms, some acute nonlymphocytic leukemias, and neuroblastomas.

As used herein, the term “anti-CD25” in reference to an antibody that specifically binds CD25 and includes reference to an antibody which is generated against CD25. In preferred embodiments, the CD25 is a primate CD25, such as human CD25. In one preferred embodiment, the antibody is generated against human CD25 synthesized by a non-primate mammal after introduction into the animal of cDNA which encodes human CD25.

The term “mesothelin” refers to a protein and fragments thereof present on the surface of some human cells and bound by, for example, the K1 antibody. Nucleic acid and amino acid sequences of mesothelin are set forth in, for example, PCT published application WO 97/25,068 and U.S. Pat. Nos. 6,083,502 and 6,153,430. See also, Chang, K. & Pastan, I., Int. J. Cancer 57:90 (1994); Chang, K. & Pastan, I., Proc. Nat'l Acad. Sci. USA 93:136 (1996); Brinkmann U., et al., Int. J. Cancer 71:638 (1997); Chowdhury, P. S., et al., Mol. Immunol. 34:9 (1997), and U.S. Pat. No. 6,809,184. Mesothelin is expressed as a precursor protein of approximately 69 kDa, that then is processed to release a 30 kDa protein, while leaving attached to the cell surface the 40 kDa glycosylphosphatidylinositol linked cell surface glycoprotein described in the Background. The 40 kDa glycoprotein is the one referred to by the term “mesothelin” herein. The nucleic acid and amino acid sequences of mesothelin have been recorded from several species, e.g., human (NM_{—}005823.4→NP_{—}005814.2; and NM_{—}013404.3→NP_{—}037536.2), mouse (NM_{—}018857.1→NP_{—}061345.1), rat (NM_{—}031658.1→NP_{—}113846.1), bovine (NM_{—}001100374.1→NP_{—}001093844).

“RFB4” refers to a mouse IgG1 monoclonal antibody that specifically binds to human CD22. RFB4 is commercially available under the name RFB4 from several sources, such as Southern Biotechnology Associates, Inc. (Birmingham Ala.; Cat. No. 9360-01), Autogen Bioclear UK Ltd. (Calne, Wilts, UK; Cat. No. AB147), Axxora LLC. (San Diego, Calif.). RFB4 is highly specific for cells of the B lineage and has no detectable cross-reactivity with other normal cell types. Li et al., Cell. Immunol. 118:85-99 (1989). The heavy and light chains of RFB4 have been cloned. See, Mansfield et al., Blood 90:2020-2026 (1997), which is incorporated herein by reference.

As used herein, “antibody” includes reference to an immunoglobulin molecule immunologically reactive with a particular antigen, and includes both polyclonal and monoclonal antibodies. The term also includes genetically engineered forms such as chimeric antibodies (e.g., humanized murine antibodies), heteroconjugate antibodies (e.g., bispecific antibodies), recombinant single chain Fv fragments (scFv), and disulfide stabilized (dsFv) Fv fragments (see, co-owned U.S. Pat. No. 5,747,654, which is incorporated herein by reference). The term “antibody” also includes antigen binding forms of antibodies (e.g., Fab′, F(abs)₂, Fab, Fv and rIgG. See also, Pierce Catalog and Handbook, 1994-1995 (Pierce Chemical Co., Rockford, Ill.); Goldsby et al., eds., Kuby, J., Immunology, 4th Ed., W.H. Freeman & Co., New York (2000).

An antibody immunologically reactive with a particular antigen can be generated by recombinant methods such as selection of libraries of recombinant antibodies in phage or similar vectors, see, e.g., Huse, et al., Science 246:1275-1281 (1989); Ward, et al., Nature 341:544-546 (1989); and Vaughan, et al., Nature Biotech. 14:309-314 (1996), or by immunizing an animal with the antigen or with DNA encoding the antigen.

Typically, an immunoglobulin has a heavy and light chain. Each heavy and light chain contains a constant region and a variable region, (the regions are also known as “domains”). Light and heavy chain variable regions contain a “framework” region interrupted by three hypervariable regions, also called “complementarity-determining regions” or “CDRs”. The extent of the framework region and CDRs have been defined. See, Kabat and Wu, supra. The sequences of the framework regions of different light or heavy chains are relatively conserved within a species. The framework region of an antibody, that is the combined framework regions of the constituent light and heavy chains, serves to position and align the CDRs in three dimensional space.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found.

References to “V_{H}” or a “VH” refer to the variable region of an immunoglobulin heavy chain, including an Fv, scFv, dsFv or Fab. References to “V_{L}” or a “VL” refer to the variable region of an immunoglobulin light chain, including of an Fv, scFv, dsFv or Fab.

The phrase “single chain Fv” or “scFv” refers to an antibody in which the variable domains of the heavy chain and of the light chain of a traditional two chain antibody have been joined to form one chain. Typically, a linker peptide is inserted between the two chains to allow for proper folding and creation of an active binding site.

The phrase “disulfide bond” or “cysteine-cysteine disulfide bond” refers to a covalent interaction between two cysteines in which the sulfur atoms of the cysteines are oxidized to form a disulfide bond. The average bond energy of a disulfide bond is about 60 kcal/mol compared to 1-2 kcal/mol for a hydrogen bond.

The phrase “disulfide stabilized Fv” or “dsFv” refer to the variable region of an immunoglobulin in which there is a disulfide bond between the light chain and the heavy chain. In the context of this invention, the cysteines which form the disulfide bond are within the framework regions of the antibody chains and serve to stabilize the conformation of the antibody. Typically, the antibody is engineered to introduce cysteines in the framework region at positions where the substitution will not interfere with antigen binding.

The term “linker peptide” includes reference to a peptide within an antibody binding fragment (e.g., Fv fragment) which serves to indirectly bond the variable domain of the heavy chain to the variable domain of the light chain.

The term “parental antibody” means any antibody of interest which is to be mutated or varied to obtain antibodies or fragments thereof which bind to the same epitope as the parental antibody, but with higher affinity.

The term “hotspot” means a portion of a nucleotide sequence of a CDR or of a framework region of a variable domain which is a site of particularly high natural variation. Although CDRs are themselves considered to be regions of hypervariability, it has been learned that mutations are not evenly distributed throughout the CDRs. Particular sites, or hotspots, have been identified as these locations which undergo concentrated mutations. The hotspots are characterized by a number of structural features and sequences. These “hotspot motifs” can be used to identify hotspots. Two consensus sequences motifs which are especially well characterized are the tetranucleotide sequence RGYW and the serine sequence AGY, where R is A or G, Y is C or T, and W is A or T.

An “immunoconjugate” is a molecule comprised of a targeting portion, or moiety, such as an antibody or fragment thereof which retains antigen recognition capability, and an effector molecule, such as a therapeutic moiety or a detectable label.

An “immunotoxin” is an immunoconjugate in which the therapeutic moiety is a cytotoxin.

A “targeting moiety” is the portion of an immunoconjugate intended to target the immunoconjugate to a cell of interest. Typically, the targeting moiety is an antibody, a scFv, a dsFv, an Fab, or an F(ab′)₂.

A “toxic moiety” is the portion of a immunotoxin which renders the immunotoxin cytotoxic to cells of interest.

A “therapeutic moiety” is the portion of an immunoconjugate intended to act as a therapeutic agent.

The term “therapeutic agent” includes any number of compounds currently known or later developed to act as anti-neoplastics, anti-inflammatories, cytokines, anti-infectives, enzyme activators or inhibitors, allosteric modifiers, antibiotics or other agents administered to induce a desired therapeutic effect in a patient. The therapeutic agent may also be a toxin or a radioisotope, where the therapeutic effect intended is, for example, the killing of a cancer cell.

A “detectable label” means, with respect to an immunoconjugate, a portion of the immunoconjugate which has a property rendering its presence detectable. For example, the immunoconjugate may be labeled with a radioactive isotope which permits cells in which the immunoconjugate is present to be detected in immunohistochemical assays.

The term “effector moiety” means the portion of an immunoconjugate intended to have an effect on a cell targeted by the targeting moiety or to identify the presence of the immunoconjugate. Thus, the effector moiety can be, for example, a therapeutic moiety, a toxin, a radiolabel, or a fluorescent label.

The terms “effective amount” or “amount effective to” or “therapeutically effective amount” includes reference to a dosage of a therapeutic agent sufficient to produce a desired result, such as inhibiting cell protein synthesis by at least 50%, or killing the cell.

The term “toxin” includes reference to abrin, ricin, Pseudomonas exotoxin A (or “PE”), diphtheria toxin (“DT”), cholix toxin (“CT”), cholera exotoxin (“CET”), botulinum toxin, pokeweed antiviral protein or modified toxins thereof. For example, PE and DT are highly toxic compounds that typically bring about death through liver toxicity. Cytotoxins, however, can be modified into a form for use as an immunotoxin by removing the native targeting component of the toxin (e.g., domain Ia of PE or the B chain of DT) and replacing it with a different targeting moiety, such as an antibody. See, e.g., Kreitman, The AAPS Journal (2006) 8(3):E532-551 and the references cited therein. Preferred toxins inhibit protein synthesis, e.g., are ADP-ribosylating agents or ribosomal inactivating agents.

As indicated by the preceding paragraph, the term Pseudomonas exotoxin A (“PE”) as used herein includes reference to forms of PE which have been modified but which retain cytotoxic function. Thus, the PE molecule can be truncated to provide a fragment of PE which is cytotoxic but which does not bind cells, as in the fragments known as PE38 and PE40, or can have mutations which reduce non-specific binding, as in the version called “PE4E”, in which four residues are mutated to glutamic acid. Further, a portion of the PE sequence can be altered to increase toxicity, as in the form called “PE38 KDEL”, in which the C-terminal sequence of native PE is altered, or the form of PE discussed herein, in which the arginine corresponding to position 490 of the native PE sequence is replaced by alanine, glycine, valine, leucine, or isoleucine.

As used herein, the terms “Cholix toxin” or “CT” and “Cholera exotoxin” or “CET” refer to a toxin expressed by some strains of Vibrio cholerae that do not cause cholera disease. According to the article reporting the discovery of the Cholix toxin (Jorgensen, R. et al., J Biol Chem. 283(16):10671-10678 (2008)), mature cholix toxin is a 70.7 kD, 634 residue protein, FIG. 9C of PCT/US2009/046292. The Jorgensen authors deposited in the NCBI Entrez Protein database a 642-residue sequence which consists of what they termed the full length cholix toxin A chain plus, at the N-terminus an additional 8 residues, consisting of a 6 histidine tag flanked by methionine residues, presumably introduced to facilitate expression and separation of the protein. The 642-residue sequence is available on-line in the Entrez Protein database under accession number 2Q5T_A and can be converted to the 634 amino acid sequence by simply deleting the first 8 amino acids of the deposited sequence. Mature CT has four domains: Domain Ia (amino acid residues 1-269), Domain II (amino acid residues 270-386), Domain Ib (amino acid residues 387-415), and Domain III (amino acid residues 417-634).

As used herein, the terms “Cholera exotoxin” or “CET” refer to a toxin expressed by some strains of Vibrio cholerae that do not cause cholera disease and include mature CET and cytotoxic fragments thereof. Mature cholera exotoxin (CET) is a 634 amino acid residue protein whose sequence is set forth as in FIG. 9C of PCT/US2009/046292. For convenience of reference, the terms “cholera exotoxin,” and “CET” as used herein may refer to the native or mature toxin, but more commonly refer to forms in which the toxin has been modified to reduce non-specific binding, for example, by deletion of domain Ia, or otherwise improve its utility for use in immunotoxins. A CET protein may be a full-length CET protein or it may be a partial CET protein comprising one or more subdomains of a CET protein and having cytotoxic activity as described herein. Mature CET has four domains: Domain Ia (amino acid residues 1-269), Domain II (amino acid residues 270-386), Domain Ib (amino acid residues 387-415), and Domain III (amino acid residues 417-634).

The term “contacting” includes reference to placement in direct physical association.

An “expression plasmid” comprises a nucleotide sequence encoding a molecule or interest, which is operably linked to a promoter.

As used herein, “polypeptide”, “peptide” and “protein” are used interchangeably and include reference to a polymer of amino acid residues. The terms apply to amino acid polymers in which one or more amino acid residue is an artificial chemical analogue of a corresponding naturally occurring amino acid, as well as to naturally occurring amino acid polymers. The terms also apply to polymers containing conservative amino acid substitutions such that the protein remains functional.

The term “residue” or “amino acid residue” or “amino acid” includes reference to an amino acid that is incorporated into a protein, polypeptide, or peptide (collectively “peptide”). The amino acid can be a naturally occurring amino acid and, unless otherwise limited, can encompass known analogs of natural amino acids that can function in a similar manner as naturally occurring amino acids.

The amino acids and analogs referred to herein are described by shorthand designations as follows in Table A:

TABLE A
Amino Acid Nomenclature

| Name | 3-letter | 1-letter |
|---|---|---|
| Alanine | Ala | A |
| Arginine | Arg | R |
| Asparagine | Asn | N |
| Aspartic Acid | Asp | D |
| Cysteine | Cys | C |
| Glutamic Acid | Glu | E |
| Glutamine | Gln | Q |
| Glycine | Gly | G |
| Histidine | His | H |
| Homoserine | Hse | — |
| Isoleucine | Ile | I |
| Leucine | Leu | L |
| Lysine | Lys | K |
| Methionine | Met | M |
| Methionine sulfoxide | Met (O) | — |
| Methionine | Met (S—Me) | — |
| methylsulfonium | | |
| Norleucine | Nle | — |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

A “conservative substitution”, when describing a protein refers to a change in the amino acid composition of the protein that does not substantially alter the protein's activity. Thus, “conservatively modified variations” of a particular amino acid sequence refers to amino acid substitutions of those amino acids that are not critical for protein activity or substitution of amino acids with other amino acids having similar properties (e.g., acidic, basic, positively or negatively charged, polar or non-polar, etc.) such that the substitutions of even critical amino acids do not substantially alter activity. Conservative substitution tables providing functionally similar amino acids are well known in the art. The following six groups in Table B each contain amino acids that are conservative substitutions for one another:

TABLE B

1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).
See also, Creighton, PROTEINS, W.H. Freeman and Company, New York (1984).

The terms “substantially similar” in the context of a peptide indicates that a peptide comprises a sequence with at least 90%, preferably at least 95% sequence identity to the reference sequence over a comparison window of 10-20 amino acids. Percentage of sequence identity is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “conjugating,” “joining,” “bonding” or “linking” refer to making two polypeptides into one contiguous polypeptide molecule. In the context of the present invention, the terms include reference to joining an antibody moiety to an effector molecule (EM). The linkage can be either by chemical or recombinant means. Chemical means refers to a reaction between the antibody moiety and the effector molecule such that there is a covalent bond formed between the two molecules to form one molecule.

As used herein, “recombinant” includes reference to a protein produced using cells that do not have, in their native state, an endogenous copy of the DNA able to express the protein. The cells produce the recombinant protein because they have been genetically altered by the introduction of the appropriate isolated nucleic acid sequence. The term also includes reference to a cell, or nucleic acid, or vector, that has been modified by the introduction of a heterologous nucleic acid or the alteration of a native nucleic acid to a form not native to that cell, or that the cell is derived from a cell so modified. Thus, for example, recombinant cells express genes that are not found within the native (non-recombinant) form of the cell, express mutants of genes that are found within the native form, or express native genes that are otherwise abnormally expressed, underexpressed or not expressed at all.

As used herein, “nucleic acid” or “nucleic acid sequence” includes reference to a deoxyribonucleotide or ribonucleotide polymer in either single- or double-stranded form, and unless otherwise limited, encompasses known analogues of natural nucleotides that hybridize to nucleic acids in a manner similar to naturally occurring nucleotides. Unless otherwise indicated, a particular nucleic acid sequence includes the complementary sequence thereof as well as conservative variants, i.e., nucleic acids present in wobble positions of codons and variants that, when translated into a protein, result in a conservative substitution of an amino acid.

As used herein, “encoding” with respect to a specified nucleic acid, includes reference to nucleic acids which comprise the information for translation into the specified protein. The information is specified by the use of codons. Typically, the amino acid sequence is encoded by the nucleic acid using the “universal” genetic code. However, variants of the universal code, such as is present in some plant, animal, and fungal mitochondria, the bacterium Mycoplasma capricolumn (Proc. Nat'l Acad. Sci. USA 82:2306-2309 (1985), or the ciliate Macronucleus, may be used when the nucleic acid is expressed in using the translational machinery of these organisms.

The phrase “fusing in frame” refers to joining two or more nucleic acid sequences which encode polypeptides so that the joined nucleic acid sequence translates into a single chain protein which comprises the original polypeptide chains.

As used herein, “expressed” includes reference to translation of a nucleic acid into a protein. Proteins may be expressed and remain intracellular, become a component of the cell surface membrane or be secreted into the extracellular matrix or medium.

By “host cell” is meant a cell which can support the replication or expression of the expression vector. Host cells may be prokaryotic cells such as E. coli, or eukaryotic cells such as yeast, insect, amphibian, or mammalian cells.

The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence, as measured using one of the following sequence comparison algorithms or by visual inspection.

As used herein, the term “substantially identical,” in the context of two nucleic acids or polypeptides, refers to two or more sequences or subsequences that have at least 60%, more preferably 65%, even more preferably 70%, still more preferably 75%, even more preferably 80%, and most preferably 90%, 91%, 92%, 93%, 94%, 95%, 96%, 97%, 98%, 99%, or higher nucleotide or amino acid residue identity, when compared and aligned for maximum correspondence, as measured using one of the following sequence comparison algorithms or by visual inspection. Preferably, the substantial identity exists over a region of the sequences that is at least about 50 residues in length, more preferably over a region of at least about 100 residues, and most preferably the sequences are substantially identical over at least about 150 residues. In a most preferred embodiment, the sequences are substantially identical over the entire length of the coding regions.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215: 403-410 and Altschuel et al. (1977) Nucleic Acids Res. 25: 3389-3402, respectively. Software for performing BLAST analyses is publicly available through the National Center for Biotechnology Information (on the Web at “ncbi.nlm.nih.gov/”). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions, as described below.

The term “in vivo” includes reference to inside the body of the organism from which the cell was obtained. “Ex vivo” and “in vitro” means outside the body of the organism from which the cell was obtained.

The phrase “malignant cell” or “malignancy” refers to tumors or tumor cells that are invasive and/or able to undergo metastasis, i.e., a cancerous cell.

As used herein, “mammalian cells” includes reference to cells derived from mammals including humans, rats, mice, guinea pigs, chimpanzees, or macaques. The cells may be cultured in vivo or in vitro.

The term “selectively reactive” refers, with respect to an antigen, the preferential association of an antibody, in whole or part, with a cell or tissue bearing that antigen and not to cells or tissues lacking that antigen. It is, of course, recognized that a certain degree of non-specific interaction may occur between a molecule and a non-target cell or tissue. Nevertheless, selective reactivity, may be distinguished as mediated through specific recognition of the antigen. Although selectively reactive antibodies bind antigen, they may do so with low affinity. On the other hand, specific binding results in a much stronger association between the antibody and cells bearing the antigen than between the bound antibody and cells lacking the antigen. Specific binding typically results in greater than 2-fold, preferably greater than 5-fold, more preferably greater than 10-fold and most preferably greater than 100-fold increase in amount of bound antibody (per unit time) to a cell or tissue bearing CD22 as compared to a cell or tissue lacking CD22. Specific binding to a protein under such conditions requires an antibody that is selected for its specificity for a particular protein. A variety of immunoassay formats are appropriate for selecting antibodies specifically immunoreactive with a particular protein. For example, solid-phase ELISA immunoassays are routinely used to select monoclonal antibodies specifically immunoreactive with a protein. See Harlow & Lane, ANTIBODIES, A LABORATORY MANUAL, Cold Spring Harbor Publications, New York (1988), for a description of immunoassay formats and conditions that can be used to determine specific immunoreactivity.

The term “immunologically reactive conditions” includes reference to conditions which allow an antibody generated to a particular epitope to bind to that epitope to a detectably greater degree than, and/or to the substantial exclusion of, binding to substantially all other epitopes. Immunologically reactive conditions are dependent upon the format of the antibody binding reaction and typically are those utilized in immunoassay protocols or those conditions encountered in vivo. See Harlow & Lane, supra, for a description of immunoassay formats and conditions. Preferably, the immunologically reactive conditions employed in the methods of the present invention are “physiological conditions” which include reference to conditions (e.g., temperature, osmolarity, pH) that are typical inside a living mammal or a mammalian cell. While it is recognized that some organs are subject to extreme conditions, the intra-organismal and intracellular environment normally lies around pH 7 (i.e., from pH 6.0 to pH 8.0, more typically pH 6.5 to 7.5), contains water as the predominant solvent, and exists at a temperature above 0° C. and below 50° C. Osmolarity is within the range that is supportive of cell viability and proliferation.

The terms “patient,” “subject,” “individual” interchangeably refer to a mammal, for example, a human or a non-human primate, a domesticated mammal (e.g., a canine or feline), an agricultural mammal (e.g., a bovine, porcine, ovine, equine), a laboratory mammal (a mouse, rat, hamster, rabbit).

The term “co-administer” refers to the simultaneous presence of two active agents in the blood of an individual. Active agents that are co-administered can be concurrently or sequentially delivered.

As used herein, the terms “treating” and “treatment” refer to delaying the onset of, retarding or reversing the progress of, or alleviating or preventing either the disease or condition to which the term applies, or one or more symptoms of such disease or condition.

The terms “inhibiting,” “reducing,” “decreasing” with respect to tumor or cancer growth or progression refers to inhibiting the growth, spread, metastasis of a tumor or cancer in a subject by a measurable amount using any method known in the art. The growth, progression or spread of a tumor or cancer is inhibited, reduced or decreased if the tumor burden is at least about 10%, 20%, 30%, 50%, 80%, or 100% reduced in comparison to the tumor burden prior to the co-administration of a cytotoxin that inhibits protein synthesis combined with an agent that inhibits the activity of an anti-apoptotic member of the BCL-2 family, e.g., a BH3-only mimetic. In some embodiments, the growth, progression or spread of a tumor or cancer is inhibited, reduced or decreased by at least about 1-fold, 2-fold, 3-fold, 4-fold, or more in comparison to the tumor burden prior to administration of an anti-mesothelin antibody or antibody fragment.

As used herein, the phrase “consisting essentially of” refers to the genera or species of active pharmaceutical agents included in a method or composition, as well as any excipients inactive for the intended purpose of the methods or compositions.

### BRIEF DESCRIPTION OF THE DRAWINGS

FIG. 1 illustrates the response of DLD1 cells to immunotoxin treatment. The immunotoxin, HB21-PE40, was added at various concentrations as indicated, cells harvested and assayed as indicated below. A, inhibition of protein synthesis was determined by measuring the incorporation of 3H-leucine into cells 24 h posttreatment. Results are reported as cpm/well; bars, 1 SD. B, cell viability using the WST-1 reagent was assayed after 48 h. Shown are absorbance readings at 450 nm; bars, 1 SD. C, cell energy levels, by measuring ATP, were determined after 48 h. D, apoptosis was assessed by measuring caspase 3/7 activity at 48 h.

FIG. 2 illustrates that ABT-737 overcomes resistance of DLD1 cells. Immunotoxin or immunotoxin-ABT-737 combinations were added for 48 h to DLD1 cells as indicated and assayed for viability, caspase activity, or PARP cleavage. Cycloheximide (CHX) was added to inhibit protein synthesis. A, cell viability using the WST-1 reagent shows enhanced killing with ABT-737. B, caspase 3 activity in DLD1 cells treated with immunotoxin, immunotoxin plus ABT-737, or ABT-737 alone. C, caspase 3 activity following cycloheximide, cycloheximide plus ABT-737, or ABT-737. D, Western blot of DLD1 lysates following treatments with immunotoxin alone, immunotoxin plus ABT, or ABT alone. Polyvinylidene difluoride membranes were probed for PARP cleavage or the presence of Mcl-1. Equal protein amounts (about 30 μg) were loaded in each lane.

FIG. 3 illustrates that ABT-737 enhances immunotoxin activity in SKOV3 and KB3-1 cells. A, B, and C, cells were treated as indicated for 48 h and viability was assessed using a WST-1 assay. D, KB3-1 cells were treated as indicated for 24 h, cell lysates prepared and probed with antibodies to PARP, procaspase 3, Mcl-1, and tubulin.

FIG. 4 illustrates the ABT-737-mediated enhancement of KDEL (SEQ ID NO:4) ending toxins. KB3-1 cells were incubated with various concentrations of DT (A), cycloheximide (B), or an immunotoxin made with a truncated exotoxin from V. cholerae (C) and assayed for viability 48 h posttreatment. Toxin treatments were made alone or in combination with ABT-737 at the concentrations indicated. D, the SS1P immunotoxin was added to cells either alone or in combination with ABT-737 for 18 h and then assayed for inhibition of protein synthesis.

FIG. 5 illustrates that ABT-737 causes ER stress. Lysates of ABT-treated DLD1 and KB3-1 cells were probed for the ER stress marker, ATF4. Lysates were prepared after 4 h of treatment with either 10 μmol/L of ABT-737 or 10 mmol/L of DTT.

FIG. 6 illustrates that ABT-737 and ABT-263 both exhibit immunotoxin-enhancing activity. A and B, ABT-737 enhances immunotoxin activity with even a short (4 h) exposure to the combination. ABT-737 was added in combination with immunotoxins HB21-PE40 or SS1P for 4 h, cells trypsinized and replated for 6 d. Cells that survived were visualized using methylene blue as the stain. C and D, ABT-263 enhances immunotoxin activity against DLD1 cells.

FIG. 7 illustrates that 3 μM ABT-737 enhances the cytotoxic activity of PE38 in an anti-mesothelin immunotoxin against KB adenocarcinoma cells by more than 100-fold as measured using a WST-1 assay.

FIG. 8 illustrates that 10 μM ABT-737 enhances the cytotoxic activity of Pseudomonas exotoxin (PE) against KB adenocarcinoma cells by more than 100-fold as measured using a 48 hour WST-1 assay.

FIG. 9 illustrates that 10 μM ABT-737 modestly enhances the cytotoxic activity of cycloheximide (CHX) against KB adenocarcinoma cells by less than about 5-fold as measured using a 48 hour WST-1 assay.

FIG. 10 illustrates that 3 μM ABT-737 modestly enhances the cytotoxic activity of diphtheria toxin (DT) against KB adenocarcinoma cells by less than about 5-fold as measured using a 48 hour WST-1 assay.

FIG. 11 illustrates that 3 μM ABT-737 enhances the delivery of PE40 in an anti-transferrin receptor immunotoxin to the cytosol of KB adenocarcinoma cells by 10-fold as measured by inhibition of protein synthesis over 24 hours.

FIG. 12 illustrates that 3 μM ABT-737 enhances the delivery of PE40 in an anti-mesothelin immunotoxin to the cytosol of KB adenocarcinoma cells by 10-fold as measured by inhibition of protein synthesis over 24 hours.

FIG. 13 illustrates a re-plating experiment of KB adherent cells. Subjecting the cells to a combination of 3 μM ABT-737 and PE40 in an anti-transferrin receptor immunotoxin resulted in complete cell elimination after 4 hrs exposure.

FIG. 14 illustrates a re-plating experiment of KB adherent cells. Subjecting the cells to a combination of 3 μM ABT-737 and 1 μg or 10 μg CHX did not result in cell elimination after 4 hrs exposure and 6 days incubation.

FIG. 15 illustrates the effect of ABT-263 alone on Raji cells Raji—a Burkitt's Lymphoma derived cell line. The intensity of staining for Annexin V is shown on the X-axis. The y axis shows staining with a dye 7-AAD that is normally excluded from living cells.

FIG. 16 illustrates the effect BL22 alone and BL22 in combination with ABT-263 on Raji cells. The intensity of staining for Annexin V is shown on the X-axis. The y axis shows staining with a dye 7-AAD that is normally excluded from living cells.

FIG. 17 illustrates the effect of HA22 alone and HA22 an anti-CD22 PE immunotoxin (see, Clinical Cancer Research (2005) 11, 1545-1550) in combination with ABT-263 or further in combination with staurosporine, an inhibitor of protein kinase, on apoptosis of Raji cells. The intensity of staining for Annexin V is shown on the X-axis. The y axis shows staining with a dye 7-AAD that is normally excluded from living cells.

FIG. 18 illustrates the effect of HA22 alone and HA22 an anti-CD22 PE immunotoxin (see, Clinical Cancer Research (2005) 11, 1545-1550) in combination with ABT-263 on caspase activation in Raji cells.

FIG. 19 illustrates the effects of a combination therapy according to the invention on small cell lung cancer cell line H69AR in vitro. FIG. 19a shows the dose response for inhibition of protein synthesis by HB21-PE40. FIG. 19b shows the effect on cells in culture of the combination treatment (ABT263 1 μM and HB21-PE40 (10 mg/ml))) vs. each treatment alone or control.

FIG. 20 illustrates the effect of ABT737 (50 mg/kg) and HB21-PE40 (0.4 mg/kg) on H69AR tumor xenografts in Balb c athymic nude mice.

FIG. 21 shows the results of experiments evaluating how KLM1 and other pancreatic cancer cell lines, including those with minimal mesothelin expression, respond to SS1P+ABT737. All 4 lines were treated for 24 hr with SS1P (300 ng/ml), ABT737 (10 uM) and SS1P+ABT737. FACS analysis results are displayed per cell line ((FIG. 21a) and per treatment regime (FIG. 21b).

FIG. 22 sets forth the number of mesothelin sites/cell for each of the four pancreatic cancer cell lines of FIG. 21.

### DETAILED DESCRIPTION

1. Introduction

The present invention is based, in part, on the surprising discovery that combined administration to cancer cells of chimeric molecule comprising a cytotoxin moiety that inhibits protein synthesis and an inhibitor of an anti-apoptotic member of the Bcl-2 family, e.g., a BH3-only mimetic, e.g., ABT-737, produces remarkable synergy in inducing cell death in cancer cells and other aberrantly proliferating cells that can be eliminated with targeted toxins. In addition, the combination produces apoptosis (programmed cell death) in cells that are resistant to apoptosis when either the immunotoxin or the BH3-only mimetic is added alone. Combining a PE cytotoxin with a BH3-only mimetic, e.g., ABT-737 or ABT-263, unexpectedly enhanced killing of several different types of tumor cells by at least about 100-fold in comparison to exposing the tumor cells to the PE cytotoxin or the BH3-only mimetic alone. Combining a cytotoxin that inhibits protein synthesis, e.g., DT, with a BH3-only mimetic, e.g., ABT-737 or ABT-263, unexpectedly enhanced killing of several different types of tumor cells by at least about 5-fold in comparison to exposing the tumor cells to the DT cytotoxin or the BH3-only mimetic alone.

Immunotoxins inhibit protein synthesis but do not always kill cells. Apparently some cancer cells resist killing by immunotoxins in the same way they resist chemotherapy. BH3-only mimetics target the BCL2 family of proteins. High levels of BCL2 proteins are anti-apoptotic and cells that express high levels of BCL2 proteins become very difficult to kill, e.g., with an immunotoxin. There are four main BCL2 proteins, BCL2, BCL-xl, BCL-w and MCL1. Compounds that bind to and inactivate the BH3 domain on BCL2 family of proteins, including the BH3-only mimetic ABT-737, target BCL2, BCL-xl, BCL-w but not MCL1. MCL1 is a very short-lived protein, with a half-life of about 30 minutes. Immunotoxins with a cytotoxin moiety that interferes with protein production inhibit protein synthesis when delivered to target cells. When protein synthesis is shut down, MCL1 is degraded within the time period of its short half-life and is then absent from the cell. Therefore the combination of a BH3-only mimetic and a targeted immunotoxins with a toxin moiety that inhibits protein synthesis comprise an advantageous combination treatment to disable all four major BCL2 proteins, overcome resistance to killing the cancer cells, and thus eliminate cancer cells targeted by the immunotoxin component of this combination.

2. Compositions

a. Chimeric Molecule Component

i. Targeting Moiety

In a preferred embodiment, the targeting moiety is an antibody, preferably an antibody specifically binding to a surface marker on a cell. Accordingly, in some embodiments, the chimeric molecule is an immunotoxin.

In another preferred embodiment, the targeting moiety is an antibody fragment, preferably an antibody fragment specifically binding to a surface marker on a cell. A preferred antibody fragment is a single chain Fv. Herein the construction and characterization of cytotoxin-based immunotoxins wherein the cytotoxin is fused to a scFv are described. Other preferred antibody fragments to which a toxin or cytotoxic fragment can be fused include Fab, Fab′, F(ab′)2, Fv fragment, a helix-stabilized antibody, a diabody, a disulfide stabilized antibody, and a domain antibody.

The fusion of a cytotoxin to an antibody or antibody fragment can be either to the N-terminus or C-terminus of the antibody or antibody fragment. Such fusion typically is accomplished employing recombinant DNA technologies.

In another preferred embodiment, the targeting moiety is a ligand specifically binding to a receptor on a cell surface. The ligand can be any ligand which binds to a cell surface marker. A preferred ligand is VEGF, Fas, TRAIL, a cytokine, a hormone. Other preferred ligands include, but are not limited to, TGFα, IL-2, IL15, IL4, IL13, etc.

ii. Target Cell Surface Markers

The targeting component of the chimeric molecule can be against a cell surface marker. The cell surface marker can be a protein or a carbohydrate. The cell surface antigen can be a tumor associated antigen. Preferably, the cell surface marker is exclusively expressed, preferentially expressed or expressed at clinically relevant higher levels on cancer cells or other aberrantly proliferating cells. Cell surface antigens that are targets for chimeric molecules are well known in the art, and summarized, e.g., in Mufson, Front Biosci (2006) 11:337-43; Frankel, Clin Cancer Res (2000) 6:326-334 and Kreitman, AAPS Journal (2006) 8(3):E532-E551.

Exemplary cell surface marker targets include cell surface receptors. Cell surface receptor that can be targeted using a toxin of the present invention include, but are not limited to, transferrin receptor, EGF receptor, CD19, CD22, CD25, CD21, CD79, mesothelin and cadherin. Additional cell surface antigens subject to targeted immunotoxin therapy include without limitation MUC1, MAGE, PRAME, CEA, PSA, PSMA, GM-CSFR, CD56, HER2/neu, erbB-2, CD5, CD7. Other cell surface tumor associated antigens are known and find use as targets.

The antigen targets can be found on numerous different types of cancer cells, including without limitation neuroblastoma, intestine carcinoma, rectum carcinoma, colon carcinoma, familiary adenomatous polyposis carcinoma, hereditary non-polyposis colorectal cancer, esophageal carcinoma, labial carcinoma, larynx carcinoma, hypopharynx carcinoma, tong carcinoma, salivary gland carcinoma, gastric carcinoma, adenocarcinoma, medullary thyroid carcinoma, papillary thyroid carcinoma, follicular thyroid carcinoma, anaplastic thyroid carcinoma, renal carcinoma, kidney parenchym carcinoma, ovarian carcinoma, cervix carcinoma, uterine corpus carcinoma, endometrial carcinoma, chorion carcinoma, pancreatic carcinoma, prostate carcinoma, testis carcinoma, breast carcinoma, urinary carcinoma, melanoma, brain tumors, glioblastoma, astrocytoma, meningioma, medulloblastoma, peripheral neuroectodermal tumors, Hodgkin lymphoma, non-Hodgkin lymphoma, Burkitt lymphoma, acute lymphatic leukemia (ALL), chronic lymphatic leukemia (CLL), acute myeloid leukemia (AML), chronic myeloid leukemia (CML), adult T-cell leukemia lymphoma, hepatocellular carcinoma, gall bladder carcinoma, bronchial carcinoma, small cell lung carcinoma, non-small cell lung carcinoma, multiple myeloma, basalioma, teratoma, retinoblastoma, choroids melanoma, seminoma, rhabdomyosarcoma, craniopharyngeoma, osteosarcoma, chondrosarcoma, myosarcome, liposarcoma, fibrosarcoma, Ewing sarcoma, and plasmocytoma.

In some embodiments, the cell surface marker is mesothelin. Exemplary cancers whose growth, spread and/or progression can be reduced or inhibited by targeting mesothelin include ovarian cancer, mesothelioma, non-small cell lung cancer, lung adenocarcinoma, fallopian tube cancer, head and neck cancer, cervical cancer and pancreatic cancer.

In some embodiments, the cell surface marker is CD22. Exemplary cancers whose growth, spread and/or progression can be reduced or inhibited by targeting CD22 include hairy cell leukemia, chronic lymphocytic leukemia (CLL), prolymphocytic leukemia (PLL), non-Hodgkin's lymphoma, Small Lymphocytic Lymphoma (SLL) and acute lymphatic leukemia (ALL).

In some embodiments, the cell surface marker is CD25. Exemplary cancers whose growth, spread and/or progression can be reduced or inhibited by targeting CD25 include leukemias and lymphomas, including hairy cell leukemia, and Hodgkin's lymphoma.

In some embodiments, the cell surface marker is a carbohydrate, e.g., Lewis Y antigen. Exemplary cancers whose growth, spread and/or progression can be reduced or inhibited by targeting Lewis Y antigen include bladder cancer, breast cancer, ovarian cancer, colorectal cancer, esophageal cancer, gastric cancer, lung cancer and pancreatic cancer.

In some embodiments, the cell surface marker is CD33. Exemplary cancers whose growth, spread and/or progression can be reduced or inhibited by targeting CD33 include acute myeloid leukemia (AML), chronic myelomonocytic leukemia (CML), and myeloproliferative disorders.

iii. Cytotoxins that Interfere with Protein Synthesis

Cytotoxins for use in the present invention inhibit protein synthesis. A number of plant and bacterial toxins have been studied for their suitability as the toxin component of immunotoxins. For example, the bacterial toxin known as Pseudomonas exotoxin A (“PE”) has been studied for two decades as a toxin for use in chimeric molecules, e.g., immunotoxins. Typically, PE has been truncated or mutated to reduce its non-specific toxicity while retaining its toxicity to cells to which it is targeted by the antibody portion of the immunotoxin. Over the years, numerous mutated and truncated forms of PE have been developed and clinical trials employing some of them are ongoing.

Bacterial protein toxins are well known in the art, and are discussed in such sources as Burns, D., et al., eds., BACTERIAL PROTEIN TOXINS, ASM Press, Herndon Va. (2003), Aktories, K. and Just, I., eds., BACTERIAL PROTEIN TOXINS (HANDBOOK OF EXPERIMENTAL PHARMACOLOGY), Springer-Verlag, Berlin, Germany (2000), and Alouf, J. and Popoff, M., eds., THE COMPREHENSIVE SOURCEBOOK OF BACTERIAL PROTEIN TOXINS, Academic Press, Inc., San Diego, Calif. (3rd Ed., 2006).

In some embodiments, the cytotoxin moiety is an ADP-ribosyltransferase. Pseudomonas exotoxin A (“PE”), diphtheria toxin (“DT”) and cholix toxin (“CT”), cholera exotoxin (“CET”) irreversibly ribosylate elongation factor 2 (“EF-2”) in eukaryotic cells, causing the death of affected cells by inhibiting their ability to synthesize proteins. Since EF-2 is essential for protein synthesis in eukaryotic cells, inactivation of the EF-2 in a eukaryotic cell causes death of the cell. The sequences and structure of PE, DT, CT and CET are well known in the art. Mutated forms of DT suitable for use in immunotoxins are known in the art. See, e.g., U.S. Pat. Nos. 5,208,021 and 5,352,447. DT does not share significant sequence identity or structural similarity with PE. Since most persons in the developed world have been immunized against diphtheria, DT-based immunotoxins can generally only be used in compartments of the body, such as the brain, that cannot be accessed by antibodies.

ADP-ribosylating cytotoxins and variants thereof that find use are described, for example, in co-pending application PCT/US2009/046292 and U.S. Patent Publ. No. 2009/0142341, the disclosures of both of which are hereby incorporated herein by reference in their entirety for all purposes.

In some embodiments, the toxin moiety is a ribosome inactivating agent, for example a shiga toxin, a ricin toxin or a pokeweed antiviral protein (PAP) toxin. Shiga toxins and ricin toxin act to inhibit protein synthesis by functioning as N-glycosidases, cleaving several nucleobases from ribosomal RNA. PAP depurinates 25S ribosomal RNA.

Ribosomal inactivating proteins are reviewed, e.g., in Stirpe and Battelli, Cell Mol Life Sci. (2006) 63(16):1850-66.

Variants of cytotoxins useful in immunotoxins are reviewed, e.g., in Kreitman, The AAPS Journal (2006) 8(3):E532-551 and the references cited therein.

In particularly preferred embodiments, the cytotoxin is a cytotoxic protein or immunotoxin comprising or engineered to comprise an endoplasmic reticulum retention sequence (e.g., REDLK (SEQ ID NO:2), REDL (SEQ ID NO:3), or KDEL (SEQ ID NO:4)). In further preferred embodiments, the protein or immunotoxin has ADP ribosylation activity and/or is an inhibitor of protein synthesis in the target cell. In other embodiments, the toxin (e.g., cytotoxic protein or immunotoxin) comprises an endoplasmic reticulum retention sequence (e.g., REDLK (SEQ ID NO:2), REDL (SEQ ID NO:3), or KDEL (SEQ ID NO:4)) and is not an inhibitor of protein synthesis or lacks ADP ribosylation activity.

1. Pseudomonas Exotoxin A

In preferred embodiments of the present invention, the toxin is a Pseudomonas exotoxin (“PE”) or a variant thereof. The term “Pseudomonas exotoxin” as used herein refers to a PE that has been modified from the native sequence to reduce or to eliminate non-specific binding. Such modifications may include, but are not limited to, elimination of domain Ia, various amino acid deletions in domains Ib, II and III, single amino acid substitutions and the addition of one or more sequences at the carboxyl terminus such as KDEL (SEQ ID NO:4) and REDL (SEQ ID NO:3). See Siegall, et al., J. Biol. Chem. 264:14256-14261 (1989). In a preferred embodiment, the cytotoxic fragment of PE retains at least 50%, preferably 75%, more preferably at least 90%, and most preferably 95% of the cytotoxicity of native PE when delivered to a cell bearing mesothelin. In a most preferred embodiment, the cytotoxic fragment, when delivered by an antibody or ligand, is more toxic than native PE.

Native Pseudomonas exotoxin A (“PE”) is an extremely active monomeric protein (molecular weight 66 kD), secreted by Pseudomonas aeruginosa, which inhibits protein synthesis in eukaryotic cells. The native 613 amino acid sequence of PE is provided in U.S. Pat. No. 5,602,095, incorporated herein by reference. The method of action is inactivation of the ADP-ribosylation and inactivation of elongation factor 2 (EF-2). The exotoxin contains three structural domains that act in concert to cause cytotoxicity. Domain Ia (amino acids 1-252) mediates cell binding. Domain II (amino acids 253-364) is responsible for translocation into the cytosol and domain III (amino acids 400-613) mediates ADP ribosylation of elongation factor 2. The function of domain Ib (amino acids 365-399) remains undefined, although a large part of it, amino acids 365-380, can be deleted without loss of cytotoxicity. See Siegall, et al., (1989), supra.

The term “PE” as used herein includes cytotoxic fragments of the native sequence, and conservatively modified variants of native PE and its cytotoxic fragments. Cytotoxic fragments of PE include those which are cytotoxic with or without subsequent proteolytic or other processing in the target cell (e.g., as a protein or pre-protein). Cytotoxic fragments and variants of PE have been investigated for years as agents for clinical use; several of these fragments and variants are described below. For convenience, residues of PE which are deleted or mutated are typically referred to in the art by their position in the 613 amino acid sequence of native PE. As noted, the 613-amino acid sequence of native PE is well known in the art.

In preferred embodiments, the PE has been modified to reduce or eliminate non-specific cell binding. Frequently, this is achieved by deleting domain Ia. as taught in U.S. Pat. No. 4,892,827, although it can also be achieved by, for example, mutating certain residues of domain Ia. U.S. Pat. No. 5,512,658, for instance, discloses that a mutated PE in which Domain Ia is present but in which the basic residues of domain Ia at positions 57, 246, 247, and 249 are replaced with acidic residues (glutamic acid, or “E”)) exhibits greatly diminished non-specific cytotoxicity. This mutant form of PE is sometimes referred to as “PE4E”.

One derivative of PE in which Domain Ia is deleted has a molecular weight of 40 kDa and is correspondingly known as PE40. See, Pai, et al., Proc. Nat'l Acad. Sci. USA 88:3358-62 (1991); and Kondo, et al., J. Biol. Chem. 263:9470-9475 (1988). Another derivative is PE25, containing the 11-residue fragment from domain II and all of domain III. In some embodiments, the derivative of PE contain only domain III.

In some embodiments, the cytotoxic fragment PE38 is employed. PE38 is a truncated PE pro-protein composed of PE amino acids 253-364 and 381-613 which is activated to its cytotoxic form upon processing within a cell (see e.g., U.S. Pat. No. 5,608,039, and Pastan et al., Biochim. Biophys. Acta 1333:C1-C6 (1997)). In some embodiments, the lysine residues at positions 590 and 606 of PE in PE38 are mutated to glutamines, while the lysine at position 613 is mutated to arginine, to create a form known as “PE38QQR.” See, e.g., Debinski and Pastan, Bioconj. Chem., 5: 40-46 (1994). This form of PE was originally developed in the course of increasing the homogeneity of immunotoxins formed by chemically coupling the PE molecules to the targeting antibodies.

In some embodiments, the cytotoxic fragment PE35 is employed. PE35 is a 35 kD carboxyl-terminal fragment of PE in which amino acid residues 1-279 have deleted and the molecule commences with a methionine residue at position 280, followed by amino acids 281-364 and 381-613 of native PE. PE35 and PE40 are disclosed, for example, in U.S. Pat. Nos. 5,602,095 and 4,892,827.

Further, several means are known for increasing the cytotoxicity of PE by altering residues in domain III from the native sequence. Studies have determined that certain amino acid sequences and repeats of these sequences could be used in place of the native sequence of residues 609-613 of PE to increase the cytotoxicity of the resulting PE compared to PE made with the native sequence (the native sequence of residues 609-613 and specific mutations that increase cytotoxicity are discussed in more detail below in the section entitled “Pseudomonas exotoxin A”. More recently, it has been determined that a substitution of glycine, alanine, valine or other residues for the arginine present at position 490 of the native PE sequence would increase cytotoxicity, with substitution of the arginine by alanine being particularly advantageous. See, e.g., U.S. Published Patent Application 2007/0189962; Bang et al., Clin Cancer Res, 11:1545-1550 (2005). While PEs of the invention using the native domain III sequence are expected to be useful by themselves, if desired the cytotoxicity of the PE can be augmented by using one or more of these substitutions or mutations. Any particular substitution or mutation can be tested to determine whether it retains adequate cytotoxicity for in vitro use and whether it has sufficiently low non-specific toxicity for in vivo use using assays known in the art, including those described in WO 2009/032954.

In some embodiments, the PE toxin is modified to remove epitopes recognized by T cells and/or B cells. The presence of epitopes or subepitopes have been mapped in domain III. Binding of antibodies which recognize those epitopes can be reduced or eliminated by substitutions of the residues normally present at certain positions. U.S. Published Patent Application 2007/0189962 demonstrated that the binding of these antibodies can be reduced by substituting an alanine, glycine, serine or glutamine for one or more amino acid residues selected from the group consisting of D403, R412, R427, E431, R432, R458, D461, R467, R505, R513, E522, R538, E548, R551, R576, K590, and L597 in a PE (the positions are made with reference to the PE sequence in WO 2009/032954). Since the presence of these residues prior to their substitution maintains an epitope or subepitope in domain III, for ease of reference, the residues at these positions can be referred to as “maintaining” the immunogenicity of their respective epitopes or subepitopes, while substituting them with alanine or the like reduces the immunogenicity of PE domain III resulting from the native epitope or subepitope. While PEs of the invention using the native domain III sequence are expected to be useful by themselves, therefore, if desired substitutions of one of more of the residues identified above can be made to reduce further the immunogenicity of the PEs of the invention. Any particular substitution or mutation can be tested to determine whether it retains adequate cytotoxicity for in vitro or in vivo use using assays known in the art, including those set forth WO 2009/032954 and in PCT/US2009/046292.

In some embodiments, the PE toxin is modified to remove amino acid segment(s) that are targets of lysosomal proteases, i.e., are lysosomal resistant (“LR”). Exemplary lysosomal resistant variants of PE are described, e.g., in Weldon, et al., Blood (2009) 113:3792-3800 and in WO 2009/032954. In some embodiments, a cytotoxic, lysosomal resistant PE fragment selected from PE25LR, PE35LR, PE38LR or PE40LR is used.

As noted above, some or all of domain 1b may be deleted, and the remaining portions joined by a linker or directly by a peptide bond. Some of the amino portion of domain II may be deleted. And, the C-terminal end may contain the native sequence of residues 609-613 (REDLK; SEQ ID NO:2), or may contain a variation found to maintain the ability of the construct to translocate into the cytosol, such as REDL (SEQ ID NO:3) or KDEL (SEQ ID NO:4), and repeats of these sequences. See, e.g., U.S. Pat. Nos. 5,854,044; 5,821,238; and 5,602,095 and WO 99/51643. While in preferred embodiments, the PE is PE4E, PE40, PE38, or PE38QQR, any form of PE in which non-specific cytotoxicity has been eliminated or reduced to levels in which significant toxicity to non-targeted cells does not occur can be used in the immunotoxins of the present invention so long as it remains capable of translocation and EF-2 ribosylation in a targeted cell.

In some preferred embodiments, the toxicity of the PE is increased by mutating the arginine (R) at position 490 of the native sequence of PE. The R is mutated to an amino acid having an aliphatic side chain that does not comprise a hydroxyl. Thus, the R can be mutated to glycine (G), alanine (A), valine (V), leucine (L), or isoleucine (I). In preferred embodiments, the substituent is G, A, or I. Alanine is the most preferred. Surprisingly, the mutation of the arginine at position 490 to alanine doubles the toxicity of the PE molecule. The discovery of this method of increasing the toxicity of PE is disclosed in co-owned international application PCT/US2004/039617, which is incorporated herein by reference.

### Conservatively Modified Variants of PE

Conservatively modified variants of PE or cytotoxic fragments thereof have at least 80% sequence similarity, preferably at least 85% sequence similarity, more preferably at least 90% sequence similarity, and most preferably at least 95% sequence similarity at the amino acid level, with the PE of interest, such as PE38 or PE40.

The term “conservatively modified variants” applies to both amino acid and nucleic acid sequences. With respect to particular nucleic acid sequences, conservatively modified variants refer to those nucleic acid sequences which encode identical or essentially identical amino acid sequences, or if the nucleic acid does not encode an amino acid sequence, to essentially identical nucleic acid sequences. Because of the degeneracy of the genetic code, a large number of functionally identical nucleic acids encode any given polypeptide. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid.

### Assaying for Cytotoxicity of PE

Pseudomonas exotoxins employed in the invention can be assayed for the desired level of cytotoxicity by assays well known to those of skill in the art. Exemplary toxicity assays are described in, e.g., WO 00/73346, Example 2. Thus, cytotoxic fragments of PE and conservatively modified variants of such fragments can be readily assayed for cytotoxicity. A large number of candidate PE molecules can be assayed simultaneously for cytotoxicity by methods well known in the art. For example, subgroups of the candidate molecules can be assayed for cytotoxicity. Positively reacting subgroups of the candidate molecules can be continually subdivided and reassayed until the desired cytotoxic fragment(s) is identified. Such methods allow rapid screening of large numbers of cytotoxic fragments or conservative

2. Diphtheria Toxin

In some embodiments, the cytotoxin moiety is a diphtheria toxin. Diphtheria toxin (“DT”) is an exotoxin secreted by Corynebacterium diphtheriae, the pathogen bacterium that causes diphtheria. “DT” refers to a protein secreted by toxigenic strains of Corynebacterium diphtheriae. It is initially synthesized as a 535 amino acid polypeptide which undergoes proteolysis to form the toxin, which is composed of two subunits, A and B, joined by a disulfide bond. The B subunit, found at the carboxyl end, is responsible for cell surface binding and translocation; the A subunit, which is present on the amino end, is the catalytic domain, and causes the ADP ribosylation of Elongation Factor 2 (“EF-2”), thereby inactivating EF-2. See generally, Uchida et al., Science 175:901-903 (1972); Uchida et al., J Biol Chem 248:3838-3844 (1973). Mutated forms of DT suitable for use in immunotoxins are known in the art. See, e.g., U.S. Pat. Nos. 5,208,021 and 5,352,447. Once again, for convenience of reference, the term “DT” as used herein refers to the native toxin, but more commonly is used to refer to forms in which the B subunit has been deleted and in which modifications have been made in the A subunit to reduce non-specific binding and toxicity.

3. Cholix Toxin (“CT”)

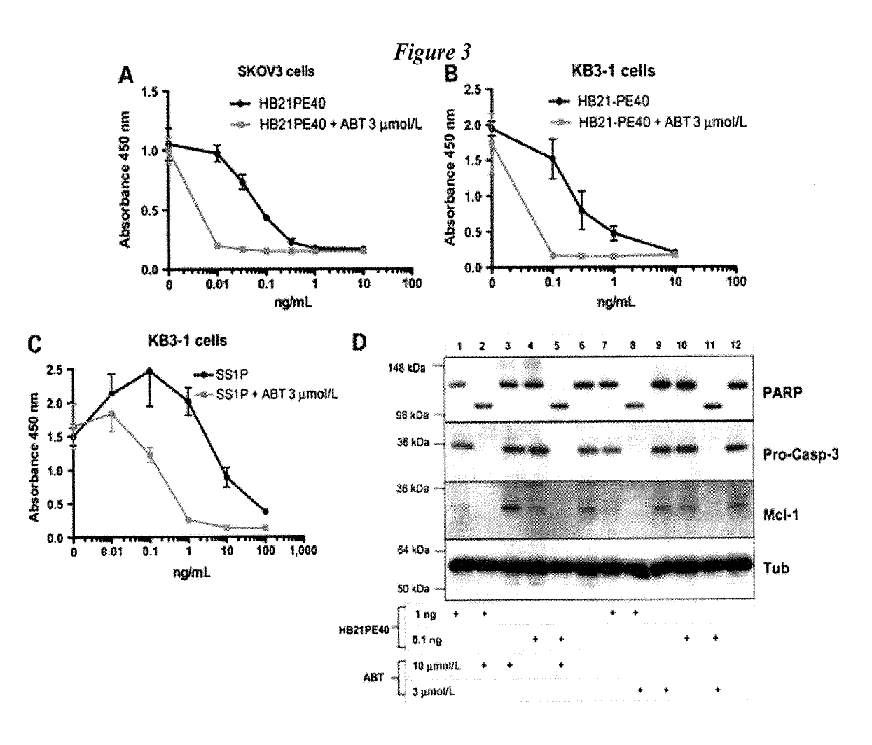

In some embodiments, the cytotoxin moiety is a cholix toxin. Jorgensen, R. et al., J Biol Chem 283(16):10671-10678 (2008) (hereafter, “Jorgensen”) recently reported that some strains of Vibrio cholerae, the causative agent of cholera, contain a ADP-ribosyltransferase, which they termed cholix toxin (also referred to herein as “CT”). Like PE, CT ribosylates EF-2. Jorgensen stated that CT's primary structure shows a 32% sequence identity with PE, and has a potential furin protease cleavage site for cellular activation, like that of PE, and contains a C-terminal KDEL sequence (SEQ ID NO:4), similar to the C-terminal sequence of PE, that likely targets the toxin to the endoplasmic reticulum of the host cell (Jorgensen, at page 10673). Jorgensen further reports that CT, like PE, is organized in three structural domains: domain Ia (residues 1-264), a receptor binding domain, a short domain Ib (residues 387-423), of unknown function, which with domain Ia comprise “a 13-stranded antiparallel β-jellyroll”, domain II (residues 265-386), a translocation domain consisting of six α-helices, and domain III, a catalytic domain with an α/β topology (Jorgensen, at page 10675). In fact, FIG. 3b of Jorgensen superpositions the structures of CT and PE, showing that the two structures are almost indistinguishable from one another.

Mature cholix toxin (CT) is a 70.7 kD, 634 residue protein. The sequence, with an eight residue leader sequence consisting of a 6-histidine tag flanked by a methionine on each side (SEQ ID NO:5), is publicly available on-line in the Entrez Protein database under accession number 2Q5T_A.

A preferred CT is a truncated version of CT in which the receptor binding domain, domain Ia, is deleted, to create a 40 kD version of CT corresponding to PE40 and referred to herein as “CT40.” Given the similarity of CT and PE, it is expected that additional variants of CT, such as a CT38 or CT35 variant, can be made that correspond to variants of PE as described in the preceding section. For example, it is anticipated that some or all of CT domain Ib can be deleted which, with the deletion of domain Ia, would create a CT variant akin to PE38. Similarly, it is anticipated that the carboxyl terminus of CT, which ends with KDELK (SEQ ID NO:1), can be varied by replacing it with one of the various C-terminal sequences mentioned above as maintaining the toxicity of PE. In preferred embodiments, if the C-terminal sequence of CT is replaced, the C-terminal sequence used as a replacement is one suitable for use in humans. In some preferred embodiments, the C-terminal sequence of CT (KDELK; SEQ ID NO:1) is replaced with the terminal sequence of PE, REDLK (SEQ ID NO:2).

Similarly, it is anticipated that the NAD domain of CT, which at least comprises amino acid residues GGEDETVIG (SEQ ID NO:6) can be varied by replacing it with another NAD domain sequence. In preferred embodiments, if the NAD domain sequence of CT is replaced, the NAD domain sequence used as a replacement is one suitable for use in humans. In some preferred embodiments, the NAD domain sequence of CT (GGEDETVIG; SEQ ID NO:6) is replaced with the NAD binding site of PE (comprising the amino acid sequence GGRLETILG; SEQ ID NO:7).

Exemplary variants of cholix toxins and immunotoxins comprising a cholix toxin that find use in the present compositions and methods are described, e.g., in co-pending application PCT/US2009/046292.

4. Cholera Exotoxin (“CET”)

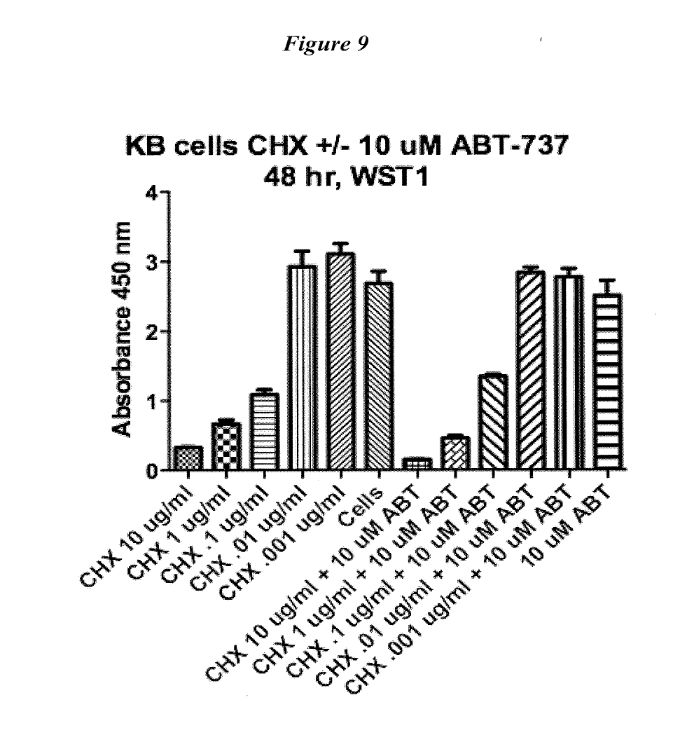

In some embodiments, the cytotoxin moiety is a cholera exotoxin (“CET”). Mature cholera exotoxin is a 634 residue protein. As shown in FIG. 9C of PCT/US2009/046292, the amino acid sequence of CET differs from that of cholix toxin in the following 14 amino acid positions: 90, (CT=H; CET=N), 213 (CT=M; CET=I), 245 (CT=V; CET=A), 266 (CT=G; CET=K), 270 (CT=S; CET=E), 295 (CT=T; CET=P), 342 (CT=D, CET=A), 345 (CT=R, CET=Q), 376 (CT=T, CET=I), 400 (CT=S; CET=P), 523, (CT=D; CET=E), 553 (CT=E; CET=R), 622 (CT=T; CET=A), and 629 (CT=R; CET=Q).

In some embodiments, the cytotoxin is a truncated version of CET in which the receptor binding domain, domain Ia, is deleted, to create a 40 kD version of CET corresponding to PE40, referred to herein as “CET40.” In one embodiment, the CET is a CET40. Given the similarity of CET and PE, it is expected that additional variants of CE such as a CET38 or CET35 variant, can be made that correspond to variants of PE as described in the preceding section. For example, it is anticipated that some or all of CET domain Ib can be deleted which, with the deletion of domain Ia, would create a CET variant akin to PE38. Similarly, it is anticipated that the carboxyl terminus of CET, which ends with KDELK (SEQ ID NO:1), can be varied by replacing it with one of the various C-terminal sequences mentioned above as maintaining the toxicity of PE. In preferred embodiments, if the C-terminal sequence of CET is replaced, the C-terminal sequence used as a replacement is one suitable for use in humans. In some preferred embodiments, the C-terminal sequence of CET (KDELK; SEQ ID NO:1) is replaced with the terminal sequence of PE, REDLK (SEQ ID NO:2).

Similarly, it is anticipated that the NAD domain of CET, which comprises at least amino acid residues GGEDETVIG (SEQ ID NO:6) can be varied by replacing it with another NAD domain sequence. In preferred embodiments, if the NAD domain sequence of CET is replaced, the NAD domain sequence used as a replacement is one suitable for use in humans. In some preferred embodiments, the NAD domain sequence of CET (GGEDETVIG; SEQ ID NO:6) is replaced with the NAD binding site of PE (comprising the amino acid sequence GGRLETILG; SEQ ID NO:7).

5. Shiga Toxin

In some embodiments, the cytotoxin moiety is a shiga toxin or a shiga-like toxin. Shiga toxins are a family of related toxins with two major groups, Stx1 and Stx2. The most common sources for Shiga toxin are the bacteria Shigella dysenteriae and the Shigatoxigenic group of Escherichia coli (STEC), which includes serotype O157:H7 and other enterohemorrhagic E. coli. Shiga toxin has two subunits—designated A and B—with a stoichiometry of ABS. The B subunit is a pentamer that binds to globotriaosylceramide (Gb3). Following this, the A subunit is internalised and cleaved into two parts. The A1 component then binds to the ribosome, disrupting protein synthesis. Stx-2 has been found to be approximately 400 times more toxic (as quantified by LD50 in mice) than Stx-1.

6. Ricin

In some embodiments, the cytotoxin moiety is ricin toxin. Ricin is a protein toxin that is extracted from the castor bean (Ricinus communis). The tertiary structure of ricin is a globular, glycosylated heterodimer of approximately 60-65 kDA, comprised of Ricin A and Ricin B chains. Ricin toxin A chain (RTA) and ricin toxin B chain (RTB) are of similar molecular weight, approximately 32 kDA and 34 kDA respectively. Ricin A Chain is an N-glycoside hydrolase composed of 267 amino acids. Ricin B Chain is a lectin composed of 262 amino acids that is able to bind terminal galactose residues on cell surfaces. RTA cleaves a glycosidic bond within the large rRNA of the 60S subunit of eukaryotic ribosomes. RTA specifically and irreversibly hydrolyses the N-glycosidic bond of the adenine residue at position 4324 (A4324) within the 28S rRNA, but leaves the phosphodiester backbone of the RNA intact. The ricin targets A4324 that is contained in a highly conserved sequence of 12 nucleotides universally found in eukaryotic ribosomes. The sequence, 5′-AGUACGAGAGGA-3′ (SEQ ID NO:8), termed the sarcin-ricin loop, is important in binding elongation factors during protein synthesis. The depurination event rapidly and completely inactivates the ribosome, resulting in toxicity from inhibited protein synthesis. A single RTA molecule in the cytosol is capable of depurinating approximately 1500 ribosomes per minute.

7. Pokeweed Antiviral Protein

In some embodiments, the cytotoxin moiety is a pokeweed antiviral protein. Pokeweed antiviral protein (PAP) is another ribosome-inactivating proteins (RIPs) that inactivate ribosomes by depurinating rRNA at a specific site.

iv. Exemplary Antibodies and Immunotoxins

Numerous antibodies for use in an immunotoxin are known in the art and find use in the present compositions and methods. Exemplary antibodies against tumor antigens include without limitation antibodies against the transferrin receptor (e.g., HB21 and variants thereof), antibodies against CD22 (e.g., RFB4 and variants thereof), antibodies against CD25 (e.g., anti-Tac and variants thereof), antibodies against mesothelin (e.g., SS1, SSP1, MN and variants thereof) and antibodies against Lewis Y antigen (e.g., B3 and variants thereof).

Antibodies for inclusion in an immunotoxin and that find use in the present invention have been described, e.g., in U.S. Pat. Nos. 5,242,824 (anti-transferrin receptor); 5,846,535 (anti-CD25); 5,889,157 (anti-Lewis Y); 5,981,726 (anti-Lewis Y); 5,990,296 (anti-Lewis Y); 7,081,518 (anti-mesothelin); 7,355,012 (anti-CD22 and anti-CD25); 7,368,110 (anti-mesothelin); 7,470,775 (anti-CD30); 7,521,054 (anti-CD25); 7,541,034 (anti-CD22); in U.S. Patent Publ. No. 2007/0189962 (anti-CD22), and reviewed in, e.g., Frankel, Clin Cancer Res (2000) 6:326-334 and Kreitman, AAPS Journal (2006) 8(3):E532-E551.

Numerous immunotoxins successfully used in anticancer and acute graft-versus-host disease are also known in the art, and find use in the present compositions and methods. Exemplary immunotoxins can be found, for example, on the worldwide web at clinicaltrials.gov and include without limitation LMB-2 (Anti-Tac(Fv)-PE38), BL22 and HA22 (RFB4(dsFv)-PE38), SS1P(SS1(dsFv)-PE38), HB21-PE40. Additional immunotoxins of use are described in the patents listed above and herein, and are reviewed in, e.g., Frankel, Clin Cancer Res (2000) 6:326-334 and Kreitman, AAPS Journal (2006) 8(3):E532-E551.

HA22 is a recently developed, improved form of BL22. In HA22, residues SSY in the CDR3 of the antibody variable region heavy chain (“V_{H}”) were mutated to THW. Compared to its parental antibody, RFB4, HA22 has a 5-10-fold increase in cytotoxic activity on various CD22-positive cell lines and is up to 50 times more cytotoxic to cells from patients with CLL and HCL (Salvatore, G., et al., Clin Cancer Res, 8(4):995-1002 (2002); see also, co-owned application PCT/US02/30316, International Publication WO 03/027135).

SS1P has been shown to specifically kill mesothelin expressing cell lines and to cause regressions of mesothelin expressing tumors in mice (Hassan, R. et al., Clin Cancer Res 8:3520-6 (2002); Onda, M. et al., Cancer Res 61:5070-7 (2001)). Based on these studies and appropriate safety data, 2 phase I trials with SS1P are being conducted at the National Cancer Institute in patients with mesothelin expressing cancers (Chowdhury, P. S. et al., Proc Natl Acad Sci USA 95:669-74 (1998); Hassan, R. et al., Proc Am Soc Clin Oncol 21:29a (2002)). In addition, other therapies targeting mesothelin are in preclinical development (Thomas, A. M. et al., J Exp Med 200:297-306 (2004)).

HA22-LR and SS1P-LR are lysosomal resistant variants of the HA22 and SS1P immunotoxins where cleavage clusters for lysosomal proteases have been removed. These variants are described, e.g., in Weldon, et al., Blood, (2009) 113(16):3792-800 and in WO 2009/032954.

Exemplary immunotoxins comprising a cholix toxin and cholera exotoxin that also find use in the present compositions and methods are described, e.g., in co-pending application PCT/US2009/046292.

b. Pro-Apoptotic Component

The present compositions comprise a pro-apoptotic component. The pro-apoptotic agents that find use inhibit the activity of at least one anti-apoptotic BCL2 family member protein, e.g., Bcl-2, Bcl-XL, Bcl-w, Mcl-1, CED-9, Bfl-1/A-1, and/or Bcl-B. In some embodiments, the pro-apoptotic agent mimics the activity of a BH3 only-domain protein, e.g., Bik, Bim, Bad, Bid or Egl-1. In some embodiments, the pro-apoptotic agent is a BH3-only mimetic. Exemplary inhibitors of anti-apoptotic BCL2 family member proteins include without limitation, oblimerson sodium, AT-101, Gossypol (a.k.a. BL-193), ApoG2, TW-37, ABT-263, ABT-737, GX15-070 (a.k.a., Obatoclax), HA14-1, Tetrocarcin A, chelerythrine chloride, antimycin and BHI-1 derivatives. Exemplary BH3-only mimetics include ABT-263 and ABT-737. Inhibitors of anti-apoptotic BCL2 family member proteins and BH3-only mimetics are reviewed, e.g., in Kang and Reynolds, Clin Cancer Res (2009) 15(4):1126-32; Azmi and Mohammad, J Cell Physiol (2009) 218:13-21; Lessene, et al., Nat Rev Drug Discov (2008) 7(12):989-1000; Vogler, et al., Cell Death Differ (2009) 16(3):360-7; Labi, et al, Cell Death Differ (2008) 15(6):977-87 and Zhang, et al., Drug Resist Updat. (2007) 10(6):207-17.

In some embodiments, the pro-apoptotic agent is ABT-737 or ABT-263. The chemical structure of ABT-737 is described in Oltersdorf, et al., Nature (2005) 435:677-681.

In the present compositions, the pro-apoptotic agent can be mixed with the immunotoxin or can be attached to the immunotoxin, e.g., encapsulated in a liposome that is linked to the immunotoxin.

3. Production of Immunoconjugates

Targeted toxins of the invention include, but are not limited to, molecules in which there is a covalent linkage of a toxin molecule to an antibody or other targeting agent. The choice of a particular targeting agent depends on the particular cell to be targeted. With the toxin molecules provided herein, one of skill can readily construct a variety of clones containing functionally equivalent nucleic acids, such as nucleic acids which differ in sequence but which encode the same toxin and antibody sequence. Thus, the present invention provides nucleic acids encoding antibodies and toxin conjugates and fusion proteins thereof.

1. Recombinant Methods

The nucleic acid sequences encoding the targeting moiety and cytotoxin moiety of the targeted toxin can be prepared as described herein or by any suitable method including, for example, cloning of appropriate sequences or by direct chemical synthesis by methods such as the phosphotriester method of Narang et al., Meth. Enzymol., 68:90-99 (1979); the phosphodiester method of Brown et al., Meth. Enzymol., 68:109-151 (1979); the diethylphosphoramidite method of Beaucage et al., Tetra. Lett., 22:1859-1862 (1981); the solid phase phosphoramidite triester method described by Beaucage & Caruthers, Tetra. Letts., 22(20):1859-1862 (1981), e.g., using an automated synthesizer as described in, for example, Needham-VanDevanter et al., Nucl. Acids Res., 12:6159-6168 (1984); and, the solid support method of U.S. Pat. No. 4,458,066. Chemical synthesis produces a single stranded oligonucleotide. This may be converted into double stranded DNA by hybridization with a complementary sequence, or by polymerization with a DNA polymerase using the single strand as a template. One of skill would recognize that while chemical synthesis of DNA is limited to sequences of about 100 bases, longer sequences may be obtained by the ligation of shorter sequences.

In a preferred embodiment, the nucleic acid sequences of this invention are prepared by cloning techniques. Examples of appropriate cloning and sequencing techniques, and instructions sufficient to direct persons of skill through many cloning exercises are found in Sambrook et al., MOLECULAR CLONING: A LABORATORY MANUAL (2ND ED.), Vols. 1-3, Cold Spring Harbor Laboratory (1989)), Berger and Kimmel (eds.), GUIDE TO MOLECULAR CLONING TECHNIQUES, Academic Press, Inc., San Diego Calif. (1987)), or Ausubel et al. (eds.), CURRENT PROTOCOLS IN MOLECULAR BIOLOGY, Greene Publishing and Wiley-Interscience, NY (1987). Product information from manufacturers of biological reagents and experimental equipment also provide useful information. Such manufacturers include the SIGMA chemical company (Saint Louis, Mo.), R&D systems (Minneapolis, Minn.), Pharmacia LKB Biotechnology (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersberg, Md.), Fluka Chemica-Biochemika Analytika (Fluka Chemie AG, Buchs, Switzerland), Invitrogen, San Diego, Calif., and Applied Biosystems (Foster City, Calif.), as well as many other commercial sources known to one of skill.

Nucleic acids encoding antibodies, cytotoxins or immunotoxins can be modified to form the targeted toxins of the present invention. Modification by site-directed mutagenesis is well known in the art. Nucleic acids encoding antibodies, cytotoxins or immunotoxins can be amplified by in vitro methods. Amplification methods include the polymerase chain reaction (PCR), the ligase chain reaction (LCR), the transcription-based amplification system (TAS), the self-sustained sequence replication system (3SR). A wide variety of cloning methods, host cells, and in vitro amplification methodologies are well known to persons of skill.

In a preferred embodiment, targeted toxins are prepared by inserting the cDNA which encodes an antibody or other targeting moiety of choice, such as a cytokine, into a vector which comprises the cDNA encoding a desired cytotoxin. The insertion is made so that the targeting agent (for ease of discussion, the discussion herein will assume the targeting agent is an Fv, although other targeting agents could be substituted with equal effect) and the cytotoxin are read in frame, that is in one continuous polypeptide which contains a functional Fv region and a functional cytotoxin region. In a particularly preferred embodiment, cDNA encoding a cytotoxin is ligated to a scFv so that the toxin is located at the carboxyl terminus of the scFv. In other preferred embodiments, cDNA encoding a cytotoxin is ligated to a scFv so that the toxin is located at the amino terminus of the scFv.

Once the nucleic acids encoding a cytotoxin, an antibody, or a targeted toxin are isolated and cloned, one may express the desired protein in a recombinantly engineered cell such as bacteria, plant, yeast, insect and mammalian cells. It is expected that those of skill in the art are knowledgeable in the numerous expression systems available for expression of proteins including E. coli, other bacterial hosts, yeast, and various higher eucaryotic cells such as the COS, CHO, HeLa and myeloma cell lines. No attempt to describe in detail the various methods known for the expression of proteins in prokaryotes or eukaryotes will be made. In brief, the expression of natural or synthetic nucleic acids encoding the isolated proteins of the invention will typically be achieved by operably linking the DNA or cDNA to a promoter (which is either constitutive or inducible), followed by incorporation into an expression cassette. The cassettes can be suitable for replication and integration in either prokaryotes or eukaryotes. Typical expression cassettes contain transcription and translation terminators, initiation sequences, and promoters useful for regulation of the expression of the DNA encoding the protein. To obtain high level expression of a cloned gene, it is desirable to construct expression cassettes which contain, at the minimum, a strong promoter to direct transcription, a ribosome binding site for translational initiation, and a transcription/translation terminator. For E. coli this includes a promoter such as the T7, trp, lac, or lambda promoters, a ribosome binding site and preferably a transcription termination signal. For eukaryotic cells, the control sequences can include a promoter and preferably an enhancer derived from immunoglobulin genes, SV40, cytomegalovirus, and a polyadenylation sequence, and may include splice donor and acceptor sequences. The cassettes of the invention can be transferred into the chosen host cell by well-known methods such as calcium chloride transformation or electroporation for E. coli and calcium phosphate treatment, electroporation or lipofection for mammalian cells. Cells transformed by the cassettes can be selected by resistance to antibiotics conferred by genes contained in the cassettes, such as the amp, gpt, neo and hyg genes.

One of skill would recognize that modifications can be made to a nucleic acid encoding a polypeptide (i.e., the cytotoxins described herein, including PE, DT, CT, CET) without diminishing its biological activity. Some modifications may be made to facilitate the cloning, expression, or incorporation of the targeting molecule into a fusion protein. Such modifications are well known to those of skill in the art and include, for example, termination codons, a methionine added at the amino terminus to provide an initiation, site, additional amino acids placed on either terminus to create conveniently located restriction sites, or additional amino acids (such as poly His) to aid in purification steps.

In addition to recombinant methods, the targeted toxins can also be constructed in whole or in part using standard peptide synthesis. Solid phase synthesis of the polypeptides of the present invention of less than about 50 amino acids in length may be accomplished by attaching the C-terminal amino acid of the sequence to an insoluble support followed by sequential addition of the remaining amino acids in the sequence. Techniques for solid phase synthesis are described by Barany & Merrifield, THE PEPTIDES: ANALYSIS, SYNTHESIS, BIOLOGY. VOL. 2: SPECIAL METHODS IN PEPTIDE SYNTHESIS, PART A, pp. 3-284; Merrifield et al., J. Am. Chem. Soc., 85:2149-2156 (1963), and Stewart et al., SOLID PHASE PEPTIDE SYNTHESIS, 2ND ED., Pierce Chem. Co., Rockford, Ill. (1984). Proteins of greater length may be synthesized by condensation of the amino and carboxyl termini of shorter fragments. Methods of forming peptide bonds by activation of a carboxyl terminal end (e.g., by the use of the coupling reagent N,N′-dicycylohexylcarbodiimide) are known to those of skill.

2. Purification

Once expressed, the recombinant targeted toxins can be purified as described herein or according to standard procedures of the art, including ammonium sulfate precipitation, affinity columns, column chromatography, and the like (see, generally, R. Scopes, PROTEIN PURIFICATION, Springer-Verlag, N.Y. (1982)). Substantially pure compositions of at least about 90 to 95% homogeneity are preferred, and 98 to 99% or more homogeneity are most preferred for pharmaceutical uses. Once purified, partially or to homogeneity as desired, if to be used therapeutically, the polypeptides should be substantially free of endotoxin.

Methods for expression of single chain antibodies and/or refolding to an appropriate active form, including single chain antibodies, from bacteria such as E. coli have been described and are well-known and are applicable to the antibodies of this invention. See, Buchner et al., Anal. Biochem., 205:263-270 (1992); Pluckthun, Biotechnology, 9:545 (1991); Huse et al., Science, 246:1275 (1989) and Ward et al., Nature, 341:544 (1989), all incorporated by reference herein.

Often, functional heterologous proteins from E. coli or other bacteria are isolated from inclusion bodies and require solubilization using strong denaturants, and subsequent refolding. During the solubilization step, as is well-known in the art, a reducing agent must be present to separate disulfide bonds. An exemplary buffer with a reducing agent is: 0.1 M Tris pH 8, 6 M guanidine, 2 mM EDTA, 0.3 M DTE (dithioerythritol). Reoxidation of the disulfide bonds can occur in the presence of low molecular weight thiol reagents in reduced and oxidized form, as described in Saxena et al., Biochemistry, 9: 5015-5021 (1970), incorporated by reference herein, and especially as described by Buchner et al., supra.

Renaturation is typically accomplished by dilution (e.g., 100-fold) of the denatured and reduced protein into refolding buffer. An exemplary buffer is 0.1 M Tris, pH 8.0, 0.5 M L-arginine, 8 mM oxidized glutathione, and 2 mM EDTA.

As a modification to the two chain antibody purification protocol, the heavy and light chain regions are separately solubilized and reduced and then combined in the refolding solution. A preferred yield is obtained when these two proteins are mixed in a molar ratio such that a 5-fold molar excess of one protein over the other is not exceeded. It is desirable to add excess oxidized glutathione or other oxidizing low molecular weight compounds to the refolding solution after the redox-shuffling is completed.

4. Pharmaceutical Compositions and Administration

In one aspect the present invention provides a pharmaceutical composition or a medicament comprising at least one chimeric protein of the present invention, preferably a targeted toxin, and optionally a pharmaceutically acceptable carrier. A pharmaceutical composition or medicament can be administered to a patient for the treatment of a condition, including, but not limited to, a malignant disease or cancer.

a. Formulation

Pharmaceutical compositions or medicaments for use in the present invention can be formulated by standard techniques using one or more physiologically acceptable carriers or excipients. Suitable pharmaceutical carriers are described herein and in Remington: The Science and Practice of Pharmacy, 21^{st} Ed., University of the Sciences in Philadelphia, Lippencott Williams & Wilkins (2005). The chimeric proteins of the present invention can be formulated for administration by any suitable route, including via inhalation, topically, nasally, orally, parenterally, or rectally. Thus, the administration of the pharmaceutical composition may be made by intradermal, subdermal, intravenous, intramuscular, intranasal, inhalationally, intracerebral, intratracheal, intraarterial, intraperitoneal, intravesical, intrapleural, intracoronary, subcutaneously or intratumoral injection, with a syringe or other devices. Transdermal administration is also contemplated, as are inhalation or aerosol administration. Tablets and capsules can be administered orally, rectally or vaginally.

The compositions for administration will commonly comprise a solution of the chimeric protein, preferably a targeted toxin, dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of fusion protein in these formulations can vary widely, and will be selected primarily based on fluid volumes, viscosities, body weight and the like in accordance with the particular mode of administration selected and the patient's needs.

The targeted toxin compositions of this invention are suited for parenteral administration, including intravenous administration or administration into a body cavity.

The chimeric proteins, preferably targeted toxins, of the present invention can be formulated for parenteral administration by injection, for example by bolus injection or continuous infusion. Formulations for injection can be presented in unit dosage form, for example, in ampoules or in multi-dose containers, with an added preservative. Injectable compositions are preferably aqueous isotonic solutions or suspensions, and suppositories are preferably prepared from fatty emulsions or suspensions. The compositions may be sterilized and/or contain adjuvants, such as preserving, stabilizing, wetting or emulsifying agents, solution promoters, salts for regulating the osmotic pressure and/or buffers. Alternatively, the active ingredient can be in powder form for constitution with a suitable vehicle, for example, sterile pyrogen-free water, before use. In addition, they may also contain other therapeutically valuable substances. The compositions are prepared according to conventional mixing, granulating or coating methods, respectively, and contain about 0.1 to 75%, preferably about 1 to 50%, of the active ingredient.

Controlled release parenteral formulations of the targeted toxin compositions of the present invention can be made as implants, oily injections, or as particulate systems. For a broad overview of protein delivery systems see, Banga, A. J., THERAPEUTIC PEPTIDES AND PROTEINS: FORMULATION, PROCESSING, AND DELIVERY SYSTEMS, Technomic Publishing Company, Inc., Lancaster, Pa., (1995) incorporated herein by reference. Particulate systems include microspheres, microparticles, microcapsules, nanocapsules, nanospheres, and nanoparticles. Microcapsules contain the therapeutic protein as a central core. In microspheres the therapeutic is dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously. Microparticles are typically around 100 μm in diameter and are administered subcutaneously or intramuscularly. See, e.g., Kreuter J., COLLOIDAL DRUG DELIVERY SYSTEMS, J. Kreuter, ed., Marcel Dekker, Inc., New York, N.Y., pp. 219-342 (1994); and Tice & Tabibi, TREATISE ON CONTROLLED DRUG DELIVERY, A. Kydonieus, ed., Marcel Dekker, Inc. New York, N.Y., pp. 315-339 (1992), both of which are incorporated herein by reference.

Polymers can be used for ion-controlled release of targeted toxin compositions of the present invention. Various degradable and nondegradable polymeric matrices for use in controlled drug delivery are known in the art (Langer R., Accounts Chem. Res., 26:537-542 (1993)). For example, the block copolymer, polaxamer 407 exists as a viscous yet mobile liquid at low temperatures but forms a semisolid gel at body temperature. It has shown to be an effective vehicle for formulation and sustained delivery of recombinant interleukin-2 and urease (Johnston et al., Pharm. Res., 9:425-434 (1992); and Pec et al., J. Parent. Sci. Tech., 44(2):58-65 (1990)). Alternatively, hydroxyapatite has been used as a microcarrier for controlled release of proteins (Ijntema et al., Int. J. Pharm., 112:215-224 (1994)). In yet another aspect, liposomes are used for controlled release as well as drug targeting of the lipid-capsulated drug (Betageri et al., LIPOSOME DRUG DELIVERY SYSTEMS, Technomic Publishing Co., Inc., Lancaster, Pa. (1993)). Numerous additional systems for controlled delivery of therapeutic proteins are known. See, e.g., U.S. Pat. Nos. 5,055,303, 5,188,837, 4,235,871, 4,501,728, 4,837,028 4,957,735 and 5,019,369, 5,055,303; 5,514,670; 5,413,797; 5,268,164; 5,004,697; 4,902,505; 5,506,206, 5,271,961; 5,254,342 and 5,534,496, each of which is incorporated herein by reference.

Suitable formulations for transdermal application include an effective amount of a composition of the present invention with a carrier. Preferred carriers include absorbable pharmacologically acceptable solvents to assist passage through the skin of the host. For example, transdermal devices are in the form of a bandage comprising a backing member, a reservoir containing the composition optionally with carriers, optionally a rate controlling barrier to deliver the composition to the skin of the host at a controlled and predetermined rate over a prolonged period of time, and means to secure the device to the skin. Matrix transdermal formulations may also be used.

Suitable formulations for topical application, e.g., to the skin and eyes, are preferably aqueous solutions, ointments, creams or gels well-known in the art. Such may contain solubilizers, stabilizers, tonicity enhancing agents, buffers and preservatives.

For oral administration, a pharmaceutical composition or a medicament can take the form of, for example, a tablet or a capsule prepared by conventional means with a pharmaceutically acceptable excipient. Preferred are tablets and gelatin capsules comprising the active ingredient, i.e., a composition of the present invention, together with (a) diluents or fillers, e.g., lactose, dextrose, sucrose, mannitol, sorbitol, cellulose (e.g., ethyl cellulose, microcrystalline cellulose), glycine, pectin, polyacrylates and/or calcium hydrogen phosphate, calcium sulfate, (b) lubricants, e.g., silica, talcum, stearic acid, its magnesium or calcium salt, metallic stearates, colloidal silicon dioxide, hydrogenated vegetable oil, corn starch, sodium benzoate, sodium acetate and/or polyethyleneglycol; for tablets also (c) binders, e.g., magnesium aluminum silicate, starch paste, gelatin, tragacanth, methylcellulose, sodium carboxymethylcellulose, polyvinylpyrrolidone and/or hydroxypropyl methylcellulose; if desired (d) disintegrants, e.g., starches (e.g., potato starch or sodium starch), glycolate, agar, alginic acid or its sodium salt, or effervescent mixtures; (e) wetting agents, e.g., sodium lauryl sulphate, and/or (f) absorbents, colorants, flavors and sweeteners.

Tablets may be either film coated or enteric coated according to methods known in the art. Liquid preparations for oral administration can take the form of, for example, solutions, syrups, or suspensions, or they can be presented as a dry product for constitution with water or other suitable vehicle before use. Such liquid preparations can be prepared by conventional means with pharmaceutically acceptable additives, for example, suspending agents, for example, sorbitol syrup, cellulose derivatives, or hydrogenated edible fats; emulsifying agents, for example, lecithin or acacia; non-aqueous vehicles, for example, almond oil, oily esters, ethyl alcohol, or fractionated vegetable oils; and preservatives, for example, methyl or propyl-p-hydroxybenzoates or sorbic acid. The preparations can also contain buffer salts, flavoring, coloring, and/or sweetening agents as appropriate. If desired, preparations for oral administration can be suitably formulated to give controlled release of the active composition.

For administration by inhalation the chimeric protein, preferably an antibody and/or targeted toxin may be conveniently delivered in the form of an aerosol spray presentation from pressurized packs or a nebulizer, with the use of a suitable propellant, for example, dichlorodifluoromethane, trichlorofluoromethane, dichlorotetrafluoroethane, 1,1,1,2-tetrafluorethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the chimeric protein, preferably an antibody and/or targeted toxin and a suitable powder base, for example, lactose or starch.

The compositions can also be formulated in rectal compositions, for example, suppositories or retention enemas, for example, containing conventional suppository bases, for example, cocoa butter or other glycerides.

Furthermore, the compositions can be formulated as a depot preparation. Such long-acting formulations can be administered by implantation (for example, subcutaneously or intramuscularly) or by intramuscular injection. Thus, for example, the composition can be formulated with suitable polymeric or hydrophobic materials (for example as an emulsion in an acceptable oil) or ion exchange resins, or as sparingly soluble derivatives, for example, as a sparingly soluble salt.

The compositions can, if desired, be presented in a pack or dispenser device that can contain one or more unit dosage forms containing the active ingredient. The pack can, for example, comprise metal or plastic foil, for example, a blister pack. The pack or dispenser device can be accompanied by instructions for administration.

b. Dosage

In one embodiment of the present invention, a pharmaceutical composition or medicament is administered to a patient at a therapeutically effective dose to prevent, treat, or control a disease or malignant condition, such as cancer. The pharmaceutical composition or medicament is administered to a patient in an amount sufficient to elicit an effective therapeutic or diagnostic response in the patient. An effective therapeutic or diagnostic response is a response that at least partially arrests or slows the symptoms or complications of the disease or malignant condition. An amount adequate to accomplish this is defined as “therapeutically effective dose.”

The dosage of chimeric proteins, preferably targeted toxins, or compositions administered is dependent on the species of warm-blooded animal (mammal), the body weight, age, individual condition, surface area of the area to be treated and on the form of administration. The size of the dose also will be determined by the existence, nature, and extent of any adverse effects that accompany the administration of a particular compound in a particular subject. A unit dosage for administration to a mammal of about 50 to 70 kg may contain between about 5 and 500 mg of the active ingredient. Typically, a dosage of the compound of the present invention, is a dosage that is sufficient to achieve the desired effect.

Optimal dosing schedules can be calculated from measurements of chimeric protein, preferably targeted toxin, accumulation in the body of a subject. In general, dosage is from 1 ng to 1,000 mg per kg of body weight and may be given once or more daily, weekly, monthly, or yearly. Persons of ordinary skill in the art can easily determine optimum dosages, dosing methodologies and repetition rates. One of skill in the art will be able to determine optimal dosing for administration of a chimeric protein, preferably a targeted toxin, to a human being following established protocols known in the art and the disclosure herein.

Optimum dosages, toxicity, and therapeutic efficacy of compositions may vary depending on the relative potency of individual compositions and can be determined by standard pharmaceutical procedures in cell cultures or experimental animals, for example, by determining the LD50 (the dose lethal to 50% of the population) and the ED50 (the dose therapeutically effective in 50% of the population). The dose ratio between toxic and therapeutic effects is the therapeutic index and can be expressed as the ratio, LD₅₀/ED₅₀. Compositions that exhibit large therapeutic indices are preferred. While compositions that exhibit toxic side effects can be used, care should be taken to design a delivery system that targets such compositions to the site of affected tissue to minimize potential damage to normal cells and, thereby, reduce side effects.

The data obtained from, for example, animal studies (e.g. rodents and monkeys) can be used to formulate a dosage range for use in humans. The dosage of compounds of the present invention lies preferably within a range of circulating concentrations that include the ED₅₀ with little or no toxicity. The dosage can vary within this range depending upon the dosage form employed and the route of administration. For any composition for use in the methods of the invention, the therapeutically effective dose can be estimated initially from cell culture assays. A dose can be formulated in animal models to achieve a circulating plasma concentration range that includes the IC₅₀ (the concentration of the test compound that achieves a half-maximal inhibition of symptoms) as determined in cell culture. Such information can be used to more accurately determine useful doses in humans. Levels in plasma can be measured, for example, by high performance liquid chromatography (HPLC). In general, the dose equivalent of a chimeric protein, preferably a targeted toxin is from about 1 ng/kg to 100 mg/kg for a typical subject.

A typical targeted toxin composition of the present invention for intravenous administration would be about 0.1 to 10 mg per patient per day. Dosages from 0.1 up to about 100 mg per patient per day may be used. Actual methods for preparing administrable compositions will be known or apparent to those skilled in the art and are described in more detail in such publications as Remington: The Science and Practice of Pharmacy, 21^{st} Ed., University of the Sciences in Philadelphia, Lippencott Williams & Wilkins (2005).

Exemplary doses of the compositions described herein, include milligram or microgram amounts of the composition per kilogram of subject or sample weight (e.g., about 1 microgram per-kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a composition depend upon the potency of the composition with respect to the desired effect to be achieved. When one or more of these compositions is to be administered to a mammal, a physician, veterinarian, or researcher may, for example, prescribe a relatively low dose at first, subsequently increasing the dose until an appropriate response is obtained. In addition, it is understood that the specific dose level for any particular mammal subject will depend upon a variety of factors including the activity of the specific composition employed, the age, body weight, general health, gender, and diet of the subject, the time of administration, the route of administration, the rate of excretion, any drug combination, and the degree of expression or activity to be modulated.

In one embodiment of the present invention, a pharmaceutical composition or medicament comprising a chimeric protein, preferably a targeted toxin, of the present invention is administered, e.g., in a daily dose in the range from about 1 mg of compound per kg of subject weight (1 mg/kg) to about 1 g/kg. In another embodiment, the dose is a dose in the range of about 5 mg/kg to about 500 mg/kg. In yet another embodiment, the dose is about 10 mg/kg to about 250 mg/kg. In another embodiment, the dose is about 25 mg/kg to about 150 mg/kg. A preferred dose is about 10 mg/kg. The daily dose can be administered once per day or divided into subdoses and administered in multiple doses, e.g., twice, three times, or four times per day. However, as will be appreciated by a skilled artisan, compositions described herein may be administered in different amounts and at different times. The skilled artisan will also appreciate that certain factors may influence the dosage and timing required to effectively treat a subject, including but not limited to the severity of the disease or malignant condition, previous treatments, the general health and/or age of the subject, and other diseases present. Moreover, treatment of a subject with a therapeutically effective amount of a composition can include a single treatment or, preferably, can include a series of treatments.

Exemplary doses of ABT-263 are 100-500 mg daily doses as needed. ABT-263 can be administered at a concentration of about 25 mg/mL to about 50 mg/mL. Exemplary doses of ABT-737 are about 50-200 mg/kg, for example, about 100 mg/kg daily doses.

Following successful treatment, it may be desirable to have the subject undergo maintenance therapy to prevent the recurrence of the disease or malignant condition treated.

c. Administration

The compositions of the present invention can be administered for therapeutic treatments. In therapeutic applications, compositions are administered to a patient suffering from a disease or malignant condition, such as cancer, in an amount sufficient to cure or at least partially arrest the disease and its complications. An amount adequate to accomplish this is defined as a “therapeutically effective dose.” Amounts effective for this use will depend upon the severity of the disease and the general state of the patient's health. An effective amount of the compound is that which provides either subjective relief of a symptom(s) or an objectively identifiable improvement as noted by the clinician or other qualified observer.

Determination of an effective amount is well within the capability of those skilled in the art, especially in light of the detailed disclosure provided herein. Generally, an efficacious or effective amount of an immunoconjugate is determined by first administering a low dose or small amount of the immunoconjugate, and then incrementally increasing the administered dose or dosages, adding a second or third medication as needed, until a desired effect of is observed in the treated subject with minimal or no toxic side effects.

The cytotoxin and the pro-apoptotic compound can be administered concurrently or sequentially. The cytotoxin and the pro-apoptotic compound can be administered as a mixture or separately.

Single or multiple administrations of the compositions are administered depending on the dosage and frequency as required and tolerated by the patient. In any event, the composition should provide a sufficient quantity of the proteins of this invention to effectively treat the patient. Preferably, the dosage is administered once but may be applied periodically until either a therapeutic result is achieved or until side effects warrant discontinuation of therapy. Generally, the dose is sufficient to treat or ameliorate symptoms or signs of disease without producing unacceptable toxicity to the patient.

To achieve the desired therapeutic effect, compositions may be administered for multiple days at the therapeutically effective daily dose. Thus, therapeutically effective administration of compositions to treat a disease or malignant condition described herein in a subject may require periodic (e.g., daily) administration that continues for a period ranging from three days to two weeks or longer. Typically, compositions will be administered for at least three consecutive days, often for at least five consecutive days, more often for at least ten, and sometimes for 20, 30, 40 or more consecutive days. While consecutive daily doses are a preferred route to achieve a therapeutically effective dose, a therapeutically beneficial effect can be achieved even if the compounds or compositions are not administered daily, so long as the administration is repeated frequently enough to maintain a therapeutically effective concentration of the composition in the subject. For example, one can administer a composition every other day, every third day, or, if higher dose ranges are employed and tolerated by the subject, once a week.

Among various uses of the targeted toxins of the present invention are included a variety of disease conditions caused by specific human cells that may be eliminated by the toxic action of the fusion protein. For example, the targeted cells might express a cell surface marker such as mesothelin, CD22 or CD25.

5. Methods of Using Compositions

The compositions of the present invention find use in a variety of ways. For example, combined administration of a targeted toxin and a pro-apoptotic agent finds use to (i) induce apoptosis in a cell bearing one or more surface markers (ii) inhibit unwanted growth, hyperproliferation or survival of a cell bearing one or more cell surface markers, (iii) treat a condition, such as a cancer, and (iv) provide therapy for a mammal having developed a disease caused by the presence of cells bearing one or more cell surface marker.

Any cell or tumor cell expressing one or more cell surface marker, preferably a cell surface receptor, e.g., as described herein, can be used to practice a method of the present invention. A preferred cell or tumor cell expressing a surface marker is s selected from the group consisting of neuroblastoma, intestine carcinoma, rectum carcinoma, colon carcinoma, familiary adenomatous polyposis carcinoma, hereditary non-polyposis colorectal cancer, esophageal carcinoma, labial carcinoma, larynx carcinoma, hypopharynx carcinoma, tong carcinoma, salivary gland carcinoma, gastric carcinoma, adenocarcinoma, medullary thyroid carcinoma, papillary thyroid carcinoma, follicular thyroid carcinoma, anaplastic thyroid carcinoma, renal carcinoma, kidney parenchym carcinoma, ovarian carcinoma, cervix carcinoma, uterine corpus carcinoma, endometrium carcinoma, chorion carcinoma, pancreatic carcinoma, prostate carcinoma, testis carcinoma, breast carcinoma, urinary carcinoma, melanoma, brain tumors, glioblastoma, astrocytoma, meningioma, medulloblastoma, peripheral neuroectodermal tumors, Hodgkin lymphoma, non-Hodgkin lymphoma, Burkitt lymphoma, acute lymphatic leukemia (ALL), chronic lymphatic leukemia (CLL), acute myeloid leukemia (AML), chronic myeloid leukemia (CML), adult T-cell leukemia lymphoma, hepatocellular carcinoma, gall bladder carcinoma, bronchial carcinoma, small cell lung carcinoma, non-small cell lung carcinoma, multiple myeloma, basalioma, teratoma, retinoblastoma, choroids melanoma, seminoma, rhabdomyosarcoma, craniopharyngeoma, osteosarcoma, chondrosarcoma, myosarcome, liposarcoma, fibrosarcoma, Ewing sarcoma, and plasmocytoma.

Methods of the present invention can be practiced in vitro or in vivo. When referring to a cell, it is understood that that this term also includes a population of cells, i.e., more than one cell.

a. Using Compositions for Inducing Apoptosis in a Cell Bearing One or More Cell Surface Markers

Apoptosis plays a central role in both the development and homeostasis of multicellular organisms. “Apoptosis” refers to programmed cell death and is characterized by certain cellular characteristics, such as membrane blobbing, chromatin condensation and fragmentation, formation of apoptotic bodies and a [positive “TUNEL” (terminal deoxynucleotidyl transferase-mediated UTP nick end-labeling) staining pattern. A later step in apoptotic process is the degradation of the plasma membrane, rendering apoptotic cells leaky to various dyes (e.g., propidium iodide).

Apoptosis can be induced by multiple independent signaling pathways that converge upon a final effector mechanism consisting of multiple interactions between several death receptors and their ligands, which belong to the tumor necrosis factor (TNF) receptor/ligand superfamily. The best-characterized death receptors are CD95 (“Fas”), TNFR1 (p55), death receptor 3 (DR3 or Apo3/TRAMO), DR4 and DR5 (apo2-TRAIL-R2). The final effector mechanism of apoptosis is the activation of a series of proteinases designated as caspases. The activation of these caspases results in the cleavage of a series of vital cellular proteins and cell death.

The present invention provides methods for inducing apoptosis in a cell expressing one or more cell surface marker. In one aspect, the method for inducing apoptosis in a cell comprises the step of exposing or contacting the cell expressing one or more cell surface marker, such as a cell surface receptor, to a combination of a targeted cytotoxin that inhibits protein synthesis and an pro-apoptotic agent, as described herein. In one embodiment, the composition comprises an immunotoxin with an ADP-ribosylating cytotoxin, e.g., a PE, DT, CT, CET, or variants thereof. In one embodiment, the pro-apoptotic agent is a BH3-only mimetic, e.g., ABT-737 or ABT-263. Typically, the cells are exposed to or contacted with effective amounts of the cytotoxin and the pro-apoptotic agent, wherein the contacting results in inducing apoptosis.

In another aspect of present invention, a method of inducing a tumor cell expressing one or more cell surface marker to undergo apoptosis is provided comprising the step of co-administering a chimeric protein, preferably a targeted toxin with a pro-apoptotic agent.

In a preferred embodiment, the chimeric protein is an immunotoxin with an ADP-ribosylating cytotoxin, e.g., a PE, DT, CT, CET, or variants thereof. In one embodiment, the pro-apoptotic agent is a BH3-only mimetic, e.g., ABT-737 or ABT-263.

b. Using Compositions for Inhibiting Growth, Hyperproliferation, or Survival of a Cell Bearing One or More Cell Surface Marker

It is an object of the present invention to provide improved therapeutic strategies for treatment of cancers using the combined targeted toxin and pro-apoptotic agent compositions of the invention. In one aspect of the present invention, a method for inhibiting at least one of unwanted growth, hyperproliferation, or survival of a cell is provided. This method comprises the step of contacting a cell expressing a surface marker with an effective amount of a combination of a targeted cytotoxin that inhibits protein synthesis and a proapoptotic agent, as described herein, wherein the step of contacting results in the inhibition of at least one of unwanted growth, hyperproliferation, or survival of the cell. In one embodiment, this method comprises the step of determining whether the cell expresses one or more cell surface markers, for example, a cell surface receptor.

In a preferred embodiment, the composition comprises an immunotoxin with an ADP-ribosylating cytotoxin, e.g., a PE, DT, CT, CET, or variants thereof. In one embodiment, the pro-apoptotic agent is a BH3-only mimetic, e.g., ABT-737 or ABT-263. Typically, the cells are exposed to or contacted with an effective amounts of the cytotoxin and the pro-apoptotic agent, wherein the contacting results in the inhibition of at least one of unwanted growth, hyperproliferation, or survival of the cell.

Thus, in one aspect of the present invention methods of inhibiting growth of a population of cells bearing one or more cell surface markers are provided. In a preferred embodiment, this method comprises the steps of (a) contacting a population of cells with a chimeric protein comprising (i) a targeting moiety which specifically binds at least one of the cell surface markers and (ii) a cytotoxin that inhibits protein synthesis, and (b) contacting the population of cells with an inhibitor of an anti-apoptotic BCL-2 family member protein, e.g., a BH3-only mimetic compound. Thereby the growth of the population of cells is inhibited.

Many tumors form metastasis. Thus, in another aspect of the present invention, the compositions of the present invention are used to prevent the formation of a metastasis. This method comprises the step of administering to a tumor cell a composition of the present invention wherein the administering results in the prevention of metastasis. In a preferred embodiment, the composition comprises a targeted toxin comprising an antibody against a cell surface antigen and a cytotoxin that inhibits protein synthesis in combination with a compound that inhibits an anti-apoptotic BCL-2 family member protein, e.g., a BH3-only mimetic compound. Typically, the cells are exposed to or contacted with effective amounts of the cytotoxin and the pro-apoptotic agent, wherein the contacting results in the prevention of metastasis.

In some embodiments, the pro-apoptotic agents find use in preventing, inhibiting, reducing the proliferation of lymphocytic cells, e.g., in the context of reducing the response to: a foreign antigen, graft-vs-host disease or an autoimmune disease.

c. Using Compositions for Treating Cancer

Methods of the present invention can be practiced in vitro and in vivo. Thus, in another aspect of the present invention, a method for treating a subject suffering from a cancerous condition is provided. This method comprises the step of administering to a subject having been diagnosed with a cancer a therapeutically effective amounts of the cytotoxin and the pro-apoptotic agent, as described herein, wherein the cancerous condition is characterized by unwanted growth or proliferation of a cell expressing one or more cell surface marker, and wherein the step of administering results in the treatment of the subject.

In a preferred embodiment, the composition comprises an immunotoxin with an ADP-ribosylating cytotoxin, e.g., a PE, DT, CT, CET, or variants thereof. In one embodiment, the pro-apoptotic agent is a BH3-only mimetic, e.g., ABT-737 or ABT-263. Typically, the cells are exposed to or contacted with effective amounts of the cytotoxin and the pro-apoptotic agent, wherein the contacting results in the treatment of the subject.

Compositions of the present invention can be used to treat any cancer described herein, e.g., those subject to treatment with an immunotoxin. In one embodiment of the present invention, a combination of a cytotoxin that inhibits protein synthesis and an agent that inhibits the activity of an anti-apoptotic BCL-2 family member protein is used to treat a subject suffering from a lung cancer expressing one or more cell surface marker. A lung cancer includes, but is not limited to, bronchogenic carcinoma [squamous cell, undifferentiated small cell, undifferentiated large cell, adenocarcinoma], alveolar [bronchiolar] carcinoma, bronchial adenoma, sarcoma, lymphoma, chondromatous hamartoma, mesothelioma, SCLC, and NSCLC.

In another embodiment of the present invention, a composition of the present invention is used to treat a subject suffering from a sarcoma expressing one or more cell surface marker. A sarcoma includes, but is not limited to, cancers such as angiosarcoma, fibrosarcoma, rhabdomyosarcoma, liposarcoma, myxoma, rhabdomyoma, fibroma, lipoma and teratoma.

In yet another embodiment of the present invention, a combination of a cytotoxin that inhibits protein synthesis and an agent that inhibits the activity of an anti-apoptotic BCL-2 family member protein is used to treat a subject suffering from a gastrointestinal cancer expressing one or more cell surface marker. A gastrointestinal cancer includes, but is not limited to cancers of esophagus [squamous cell carcinoma, adenocarcinoma, leiomyosarcoma, lymphoma], stomach [carcinoma, lymphoma, leiomyosarcoma], pancreas [ductal adenocarcinoma, insulinoma, glucagonoma, gastrinoma, carcinoid tumors, VIPoma], small bowel [adenocarcinoma, lymphoma, carcinoid tumors, Kaposi's sarcoma, leiomyoma, hemangioma, lipoma, neurofibroma, fibroma], and large bowel [adenocarcinoma, tubular adenoma, villous adenoma, hamartoma, leiomyoma].

In one embodiment of the present invention, a combination of a cytotoxin that inhibits protein synthesis and an agent that inhibits the activity of an anti-apoptotic BCL-2 family member protein is used to treat a subject suffering from a cancer of the genitourinary tract expressing one or more cell surface marker. Cancers of the genitourinary tract include, but are not limited to cancers of kidney [adenocarcinoma, Wilms tumor (nephroblastoma), lymphoma, leukemia, renal cell carcinoma], bladder and urethra [squamous cell carcinoma, transitional cell carcinoma, adenocarcinoma], prostate [adenocarcinoma, sarcoma], and testis [seminoma, teratoma, embryonal carcinoma, teratocarcinoma, choriocarcinoma, sarcoma, Leydig cell tumor, fibroma, fibroadenoma, adenomatoid tumors, lipoma].

In another embodiment of the present invention, a combination of a cytotoxin that inhibits protein synthesis and an agent that inhibits the activity of an anti-apoptotic BCL-2 family member protein is used to treat a subject suffering from a liver cancer expressing one or more cell surface marker. A liver cancer includes, but is not limited to, hepatocellular carcinoma, cholangiocarcinoma, hepatoblastoma, angiosarcoma, hepatocellular adenoma, and hemangioma.

In one embodiment of the present invention, a combination of a cytotoxin that inhibits protein synthesis and an agent that inhibits the activity of an anti-apoptotic BCL-2 family member protein is used to treat a subject suffering from a skin cancer expressing one or more cell surface marker. Skin cancer includes, but is not limited to, malignant melanoma, basal cell carcinoma, squamous cell carcinoma, Kaposi's sarcoma, nevi, dysplastic nevi, lipoma, angioma, dermatofibroma, keloids, and psoriasis.

In one embodiment of the present invention, a combination of a cytotoxin that inhibits protein synthesis and an agent that inhibits the activity of an anti-apoptotic BCL-2 family member protein is used to treat a subject suffering from a gynecological cancer expressing one or more cell surface marker. Gynecological cancers include, but are not limited to, cancer of uterus [endometrial carcinoma], cervix [cervical carcinoma, pre-invasive cervical dysplasia], ovaries [ovarian carcinoma (serous cystadenocarcinoma, mucinous cystadenocarcinoma, endometrioid carcinoma, clear cell adenocarcinoma, unclassified carcinoma), granulosa-theca cell tumors, Sertoli-Leydig cell tumors, dysgerminoma, malignant teratoma and other germ cell tumors], vulva [squamous cell carcinoma, intraepithelial carcinoma, adenocarcinoma, fibrosarcoma, melanoma], vagina [clear cell carcinoma, squamous cell carcinoma, sarcoma botryoides (embryonal rhabdomyosarcoma), and fallopian tubes [carcinoma].

In yet another embodiment of the present invention, a combination of a cytotoxin that inhibits protein synthesis and an agent that inhibits the activity of an anti-apoptotic BCL-2 family member protein is used to treat a subject suffering from a bone cancer expressing one or more cell surface marker. Bone cancer includes, but is not limited to, osteogenic sarcoma [osteosarcoma], fibrosarcoma, malignant fibrous histiocytoma, chondrosarcoma, Ewing's sarcoma, malignant lymphoma [reticulum cell sarcoma], multiple myeloma, malignant giant cell tumor, chordoma, osteochondroma [osteocartilaginous exostoses], benign chondroma, chondroblastoma, chondromyxoid fibroma, osteoid osteoma, and giant cell tumors.

In one embodiment of the present invention, a combination of a cytotoxin that inhibits protein synthesis and an agent that inhibits the activity of an anti-apoptotic BCL-2 family member protein is used to treat a subject suffering from a cancer of the nervous system expressing one or more cell surface marker. Cancers of the nervous system include, but are not limited to cancers of skull [osteoma, hemangioma, granuloma, xanthoma, Paget's disease of bone], meninges [meningioma, meningiosarcoma, gliomatosis], brain [astrocytoma, medulloblastoma, glioma, ependymoma, germinoma (pinealoma), glioblastoma multiforme, oligodendroglioma, schwannoma, retinoblastoma, congenital tumors], and spinal cord [neurofibroma, meningioma, glioma, sarcoma].

In one embodiment of the present invention, a combination of a cytotoxin that inhibits protein synthesis and an agent that inhibits the activity of an anti-apoptotic BCL-2 family member protein is used to treat a subject suffering from a hematologic cancer expressing one or more cell surface marker. Hematologic cancers include, but are not limited to cancer of blood [myeloid leukemia (acute and chronic), hairy cell leukemia, acute lymphoblastic leukemia, chronic lymphocytic leukemia, myeloproliferative diseases, multiple myeloma, myelodysplastic syndrome], Hodgkin's disease, and non-Hodgkin's lymphoma (malignant lymphoma).

In one embodiment of the present invention, a combination of a cytotoxin that inhibits protein synthesis and an agent that inhibits the activity of an anti-apoptotic BCL-2 family member protein is used to treat a subject suffering from a cancer mediated by mesothelin-CA125 binding interaction. Exemplary cancers whose growth, spread and/or progression are at least partially mediated by CA125/mesothelin binding include ovarian cancer, mesothelioma, non-small cell lung cancer, lung adenocarcinoma and pancreatic cancer.

In one embodiment of the present invention, a combination of a cytotoxin that inhibits protein synthesis and an agent that inhibits the activity of an anti-apoptotic BCL-2 family member protein is used to treat a subject suffering from a cancer of adrenal glands expressing one or more cell surface marker. A cancer of adrenal glands includes, but is not limited to, neuroblastoma.

Methods for treating cancer may optionally comprise one or more of the following steps: obtaining a biological sample of tissue or fluid from an individual; screening the biological sample for the expression of one or more cell surface marker, preferably a cell surface receptor, for example by contacting the biological sample with an antibody directed to the surface marker, preferably a cell surface receptor; or screening the biological sample for expression of a surface marker polynucleotide, preferably a cell surface receptor polynucleotide, for example by detecting a surface marker mRNA, preferably, a cell surface receptor mRNA. This can be done using standard technologies known in the art, e.g., Western blotting, Northern blotting or PCR.

In another embodiment of the present invention, a combination of a cytotoxin that inhibits protein synthesis and an agent that inhibits the activity of an anti-apoptotic BCL-2 family member protein is used to treat a subject suffering from a pancreatic cancer expressing one or more cell surface marker targeted by a cytotoxin. In one further embodiment therein, the cell surface marker is mesothelin. In still other embodiments of such, the cytotoxin is a PE cytotoxin attached to a mesothelin antibody. In still further embodiments, the cytotoxin is SS1P. In other embodiments of any of the above, the inhibitor is ABT-263 or ABT-737 or another pro-apoptotic agent or BH3-only mimetic. In some embodiments, the treatment with the inhibitor overcome SS1P or mesothelin-targeted cytotoxin resistance of the targeted pancreatic tumor.

In another embodiment of the present invention, a combination of a cytotoxin that inhibits protein synthesis and an agent that inhibits the activity of an anti-apoptotic BCL-2 family member protein is used to treat a subject suffering from a small lung cell cancer expressing one or more cell surface markers targeted by a cytotoxin. In one further embodiment therein, the targeted cell surface marker is transferrin. In still other embodiments of such, the cytotoxin is a PE cytotoxin attached to a transferrin antibody or other transferrin binding agent In still further embodiments, the PE cytotoxin is a PE40 (e.g., HB21-PE40). In other embodiments of any of the above, the inhibitor is ABT-263 or ABT-737 or another pro-apoptotic agent or BH3-only mimetic. In some embodiments, the combination treatment with the inhibitor and targeted cytotoxin overcomes a resistance of the targeted small cell lung tumor to the targeted cytotoxin or both of the agents.

d. Using Compositions for Treating a Subject Having Developed a Disease Caused by the Presence of Cells Bearing One or More Cell Surface Markers

Also provided is a method a method of providing therapy for a mammal having developed a disease caused by the presence or aberrant proliferation of cells preferentially bearing or overexpressing one or more cell surface markers. In a preferred embodiment, this method comprises the step of administering to said mammal a chimeric protein comprising (i) a targeting moiety which specifically binds to at least one surface marker on said cells and (ii) a cytotoxin that inhibits protein synthesis in combination with a compound that inhibits the activity of an anti-apoptotic BCL2 family member protein, e.g., a BH3-only mimetic.

In a preferred embodiment, the chimeric protein comprises an immunotoxin with an ADP-ribosylating cytotoxin, e.g., a PE, DT, CT, CET, or variants thereof. In one embodiment, the pro-apoptotic agent is a BH3-only mimetic, e.g., ABT-737 or ABT-263. Typically, the cells are exposed to or contacted with effective amounts of the cytotoxin and the pro-apoptotic agent, wherein the contacting results in the treatment of the subject.

In another embodiment, this invention provides for eliminating target cells in vitro or ex vivo using cytotoxins of the present invention in combination with an agent that inhibits the activity of an anti-apoptotic member of the BCL-2 family. For example, chimeric molecules comprising a cytotoxin that inhibits protein synthesis and a pro-apoptotic agent can be used to purge targeted cells from a population of cells in a culture. Thus, for example, cells cultured from a patient having a cancer expressing a targeted cell surface marker (e.g., CD22, CD25, mesothelin, Lewis Y) can be purged of cancer cells by contacting the culture with chimeric molecules directed against the cell surface marker of interest in combination with an pro-apoptotic agent, as described herein.

In some instances, the target cells may be contained within a biological sample. A “biological sample” as used herein is a sample of biological tissue or fluid that contains target cells and non-target cells. Such samples include, but are not limited to, tissue from biopsy, blood, and blood cells (e.g., white cells). A biological sample is typically obtained from a multicellular eukaryote, preferably a mammal such as rat, mouse, cow, dog, guinea pig, or rabbit, and more preferably a primate, such as a macaque, chimpanzee, or human. Most preferably, the sample is from a human.

6. Methods of Disease Monitoring

The invention provides methods of detecting inhibition of tumor growth in a patient suffering from or susceptible to a cancer that can be treated with a targeted toxin, e.g., a cancer with a cell surface marker. The methods are particularly useful for monitoring a course of treatment being administered to a patient using the combined cytotoxins and pro-apoptotic agents described herein. The methods can be used to monitor both therapeutic treatment on symptomatic patients and prophylactic treatment on asymptomatic patients.

The monitoring methods entail determining a baseline value of tumor burden in a patient before administering a dosage of the combined cytotoxin and pro-apoptotic agent, and comparing this with a value for the tumor burden after treatment, or with the tumor burden in a patient receiving no treatment, or with either the cytotoxin or the pro-apoptotic agent alone.

A significant decrease (i.e., greater than the typical margin of experimental error in repeat measurements of the same sample, expressed as one standard deviation from the mean of such measurements) in value of the tumor burden signals a positive treatment outcome (i.e., that administration of the combined cytotoxin and pro-apoptotic agent has blocked progression of tumor growth and/or metastasis).

In other methods, a control value (i.e., a mean and standard deviation) of tumor burden is determined for a control population or a normal population (e.g., burden=zero). Typically, the individuals in the control population have not received prior treatment. Measured values of the tumor burden in a patient after administering the combined cytotoxin and pro-apoptotic agent are then compared with the control value. A significant decrease in tumor burden relative to the control value (e.g., greater than one standard deviation from the mean) signals a positive treatment outcome. A lack of significant decrease or an increase signals a negative treatment outcome.

In other methods, a control value of tumor burden (e.g., a mean and standard deviation) is determined from a control population of individuals who have undergone treatment receiving a regimen of combined cytotoxin and pro-apoptotic agent, as described herein. Measured values of tumor burden in a patient are compared with the control value. If the measured level in a patient is not significantly different (e.g., more than one standard deviation) from the control value, treatment can be discontinued. If the tumor burden level in a patient is significantly above the control value, continued administration of agent is warranted.

In other methods, a patient who is not presently receiving treatment but has undergone a previous course of treatment is monitored for tumor burden to determine whether a resumption of treatment is required. The measured value of tumor burden in the patient can be compared with a value of tumor burden previously achieved in the patient after a previous course of treatment. A significant increase in tumor burden relative to the previous measurement (i.e., greater than a typical margin of error in repeat measurements of the same sample) is an indication that treatment can be resumed. Alternatively, the value measured in a patient can be compared with a control value (mean plus standard deviation) determined in a population of patients after undergoing a course of treatment. Alternatively, the measured value in a patient can be compared with a control value in populations of prophylactically treated patients who remain free of symptoms of disease, or populations of therapeutically treated patients who show amelioration of disease characteristics. In all of these cases, a increase in tumor burden relative to the control level (i.e., more than a standard deviation) is an indicator that treatment should be resumed in a patient.

The tissue sample for analysis is typically blood, plasma, serum, mucous, tissue biopsy, tumor, ascites or cerebrospinal fluid from the patient. The sample can analyzed for indication of neoplasia. Neoplasia or tumor burden can be detected using any method known in the art, e.g., visual observation of a biopsy by a qualified pathologist, or other visualization techniques, e.g., radiography, ultrasound, magnetic resonance imaging (MRI).

7. Kits, Containers, Devices, and Systems

For use in diagnostic, research, and therapeutic applications described above, kits and systems are also provided by the invention. Kits of the present invention will comprise a chimeric molecule comprising a targeting moiety and a cytotoxin that inhibits protein synthesis and a pro-apoptotic agent. The embodiments of the chimeric molecules and the pro-apoptotic agents are as described herein.

In addition, the kits and systems may include instructional materials containing directions (i.e., protocols) for the practice of the methods of this invention. The instructions may be present in the subject kits in a variety of forms, one or more of which may be present in the kit. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

A wide variety of kits, systems, and compositions can be prepared according to the present invention, depending upon the intended user of the kit and system and the particular needs of the user.

In a preferred embodiment of the present invention, the kit or system comprises an immunotoxin and a compound that inhibits the activity of an anti-apoptotic family member of the BCL-2 family. The immunotoxin and the pro-apoptotic agent may be provided separately or in mixtures. The immunotoxin and the pro-apoptotic agent may be provided in uniform or varying doses. Further embodiments of the immunotoxin and the pro-apoptotic agent are as described herein.

Kits with unit doses of the active composition, e.g. in oral, vaginal, rectal, transdermal, or injectable doses (e.g., for intramuscular, intravenous, or subcutaneous injection), are provided. In such kits, in addition to the containers containing the unit doses will be an informational package insert describing the use and attendant benefits of the composition in treating a disease or malignant condition. Suitable active compositions and unit doses are those described herein above.

Although the forgoing invention has been described in some detail by way of illustration and example for clarity and understanding, it will be readily apparent to one of ordinary skill in the art in light of the teachings of this invention that certain variations, changes, modifications and substitutions of equivalents may be made thereto without necessarily departing from the spirit and scope of this invention. As a result, the embodiments described herein are subject to various modifications, changes and the like, with the scope of this invention being determined solely by reference to the claims appended hereto. Those of skill in the art will readily recognize a variety of non-critical parameters that could be changed, altered or modified to yield essentially similar results. It is also to be understood that the terminology used herein is for the purpose of describing particular embodiments only, and is not intended to be limiting, since the scope of the present invention will be limited only by the appended claims.

While each of the elements of the present invention is described herein as containing multiple embodiments, it should be understood that, unless indicated otherwise, each of the embodiments of a given element of the present invention is capable of being used with each of the embodiments of the other elements of the present invention and each such use is intended to form a distinct embodiment of the present invention.

### EXAMPLES

The following examples are offered to illustrate, but not to limit, the claimed invention.

### Example 1

### APT-737 Overcomes Resistance to Immunotoxin Mediated Apoptosis and Enhances the Delivery of Pseudomonas Exotoxin-Based Proteins to the Cell Cytosol

ABT-737 (Selleck Chemicals LLC) was dissolved in DMSO at 10 mmol/L stock concentration, and stored frozen at −20° C. ABT-263 (Toronto Research Chemicals, Inc.), was dissolved in DMSO at 3 mmol/L, and stored frozen at −20° C. Velcade (bortezomib) (NIH pharmacy). HB21-PE40 and SS1P were produced as previously described by Batra J K, et al., I. Mol Cell Biol 1991; 11:2200-5 and Hassan R, et al., Clin Cancer Res 2002; 8:3520-6). HB21-CET40 was described recently (Sarnovsky R, et al., Cancer Immunol Immunother 2010; 59:737-46). DT (List Biological Laboratories) and cycloheximide (Sigma) are commercially available.

### Antibodies

PARP (BD; 556494), caspase 3 (Santa Cruz Biotechnology; 7148), Mcl-1 (Cell Signaling; 4572), tubulin (Sigma; T6074), and ATF4 (Santa Cruz Biotechnology; SC-200).

### Cells

DLD1 and SKOV3, obtained from American Type Culture Collection, were grown in RPMI 1640 plus 10% fetal bovine serum, pen-strep, and pyruvate. The KB3-1 cells, from Michael Gottesman (National Cancer Institute, Bethesda, Md.) were grown in DMEM plus 10% fetal bovine serum.

### Assays

The inhibition of protein synthesis in treated cells was assayed by the addition of 3H-leucine (2 μCi/mL) for 4 hours in 96-well plates. Filter mats and samples were used to count cells using a Wallac beta plate reader. Water-soluble tetrazolium-1 (WST-1; Roche) was added to 96-well plates at a final concentration of 10% and absorbance measured at 450 nm to measure cytotoxicity. Te CellTiter-Glo Luminescent Cell Viability Assay kit (Promega) was used to assay ATP. The Caspase-Glo kit from Promega was used to detect Caspase 3/7. Caspase 3 activity was measured using one of two fluorescent substrates Caspase 3 Fluorometric Assay Kit (R&D Systems) or Caspase 3 Fluorometric-KIT (Invitrogen). The results are reported in fluorescent units per microgram of cell protein. For short-exposure cell-killing assays: KB3-1 cells were seeded in six-well plates at a density of 5×104/mL. The next day, cells were treated for 4 hours with either immunotoxin alone, ABT-737 alone, or combinations of both as indicated. At the end of the treatment, the cells were washed with PBS, trypsinized, and re-plated and incubated for 6 more days. Finally, the cells were washed with PBS and then stained with methylene blue (0.5%) in methanol/water (50:50 by volume).

### Western Blots

Immunotoxin-treated cells in the presence or absence of ABT-737 were washed with PBS and then solubilized with radioimmunoprecipitation assay buffer. The buffer contained both protease and phosphatase inhibitors. Cell lysates were separate using Precast Trisglycine 8% to 16% gels. Donkey anti-mouse horseradish peroxidase or donkey anti-rabbit horseradish peroxidase (Jackson ImmunoResearch) were used routinely to detect primary antibodies.

### A. AST-737 Overcomes Resistance to Immunotoxin-Mediated Apoptosis in DLD1 Cells

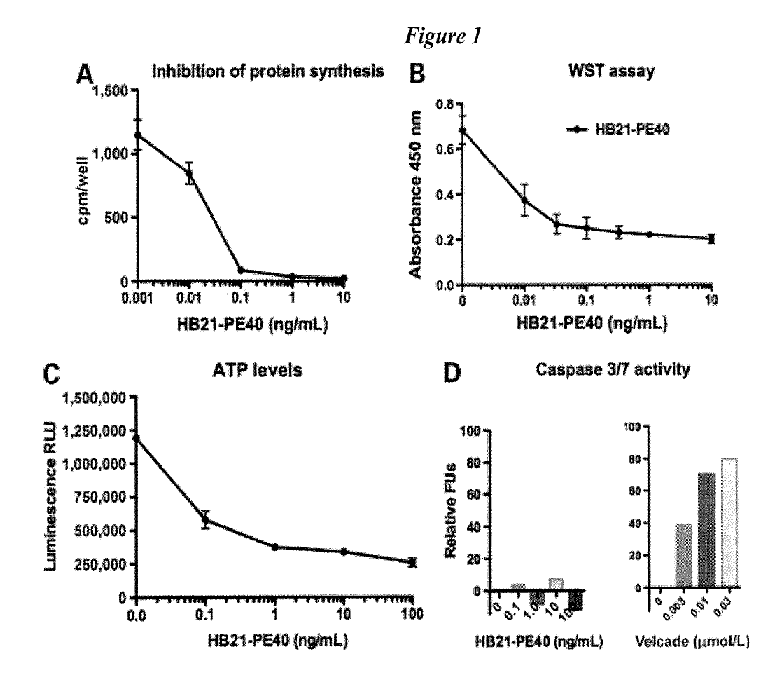

DLD1 cells are a colon cancer cell line which is susceptible to growth inhibition (FIG. 1A-C) by HB21-PE40, an immunotoxin directed to the human transferrin receptor. The inhibition exhibits a dose-response. A complete inhibition occurred at concentrations at, or exceeding, 1 ng/mL (FIG. 1A). HB21-PE40 was cytotoxic with an IC50 of about 0.1 ng/mL (FIGS. 1B and C). However, there was incomplete killing at higher concentrations (FIGS. 1B and C) as seen in a WST-1 or an ATP depletion assay. Further in this regard, about 20% of cells treated with HB21-PE40 (10 ng/mL) for 72 hours and then monitored for viability excluded trypan blue. Based on caspase 3/7 activity measurements, immunotoxin-treated DLD1 cells showed no evidence of apoptosis (FIG. 1D). However, apoptosis, as indicated by increased caspase 3/7 activity, was seen when velcade (a proteasome inhibitor) was used as a positive control (FIG. 1D). Accordingly, it is concluded that DLD1 cells are resistant to immunotoxin-mediated apoptotic death.

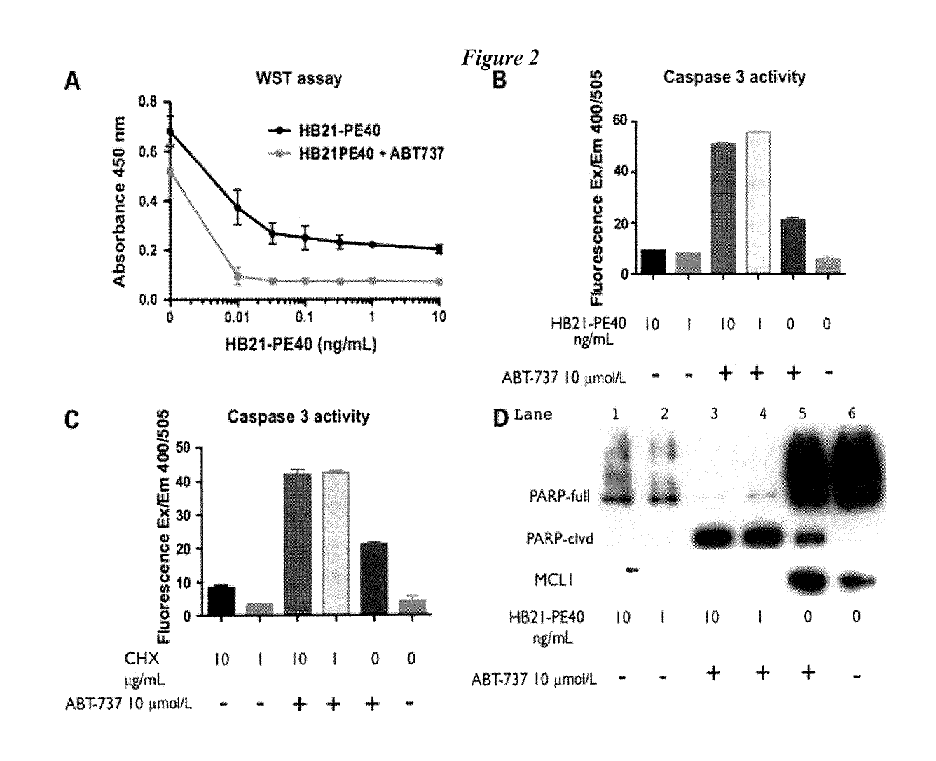

To examine the role of prosurvival Bcl-2 proteins in resistance, the effect of combining the BH-3-only mimetic, ABT-737, with immunotoxins was investigated. ABT-737 neutralizes Bcl-2, Bcl-xl, and Bcl-w but does not bind Mcl-1. As Mcl-1 has a short half-life, agents that inhibit protein synthesis, including immunotoxins, result in the loss of Mcl-1 (Adams K W, et al., J Biol Chem 2007; 282:6192-200). Accordingly, combination treatments of an immunotoxin and ABT-737 could neutralize or eliminate all the relevant prosurvival Bcl-2 family proteins. As shown in FIG. 2A, ABT-737 increased the cytotoxicity of all concentrations of the HB21-PE40 immunotoxin for DLD1 cells.

The apoptosis pathway was studied to investigate the basis for this increase. Neither immunotoxin alone (at 1 or 10 ng/mL) nor ABT-737 (at 10 μmol/L) caused a large increase in caspase 3 activity. However, the combination resulted in a substantial increase in activity compared with either treatment alone (FIG. 2B). Cycloheximide in combination with ABT-737 produced a similar result (FIG. 2C) confirming that activation of caspase 3 was primarily due to immunotoxin-mediated inhibition of protein synthesis, in combination with ABT-737.

In additional analyses of important portions of the intrinsic apoptosis pathway, radioimmunoprecipitation assay buffer extracts were probed for Mcl-1 and cleavage of PARP. Samples from immunotoxin-treated cells showed a loss of Mcl-1. Only those that were also treated with ABT-737 showed cleavage of PARP (FIG. 2D). Note the apparent increase in Mcl-1 levels in samples treated with ABT-737 alone (FIG. 2D, lane 5).

Overall, these findings show that, despite a complete reduction in protein synthesis and loss of Mcl-1, immunotoxin treatment of DLD1 cells does not by itself result in apoptosis However, when ABT-737 was added in combination, there was a significant increase in caspase 3 activity, PARP cleavage, loss of Mcl-1, and cell death (FIGS. 2B and D, lanes 3 and 4).

### B. Effect of ABT-737 on Other Cell Lines

To demonstrate a broader utility of immunotoxin-ABT-737 combinations, we tested other cell lines with other immunotoxins. Combinations of ABT-737 and HB21-PE40 were added to SKOV3. This ovarian cancer cell line is thought to be resistant to certain toxin-based agents (Morimoto H, et al., J Immunol 1991; 147:2609-16). As seen with DLD1 cells, immunotoxin and ABT-737 combinations enhanced killing by about 20-fold over the addition of immunotoxin alone (FIG. 3A). The cervical cancer cell line KB3-1 (an immunotoxin-sensitive cell line) was also tested with immunotoxins. These toxins either targeted the transferrin receptor (FIG. 3B) or mesothelin (FIG. 3C). There was a about a 20-fold greater toxicity when ABT-737 was present compared with immunotoxin alone. KB3-1 cells were also examined and the analysis was to Western blots of various apoptosis-related proteins after treatments (for 24 hours) with either the single agents or their combinations. ABT-737 treatment resulted in PARP cleavage, Mcl-1 degradation, and loss of procaspase 3 (see, FIG. 3D). Upon extending the treatments to 48 hours, immunotoxin alone resulted in PARP and procaspase 3 cleavage (data not shown). Accordingly, while KB3-1 cells were not resistant to immunotoxin induced apoptosis, the cell death evidences sooner with ABT-737 treatment.

C. ABT-737 Specifically Enhances Toxin Translocation from the ER

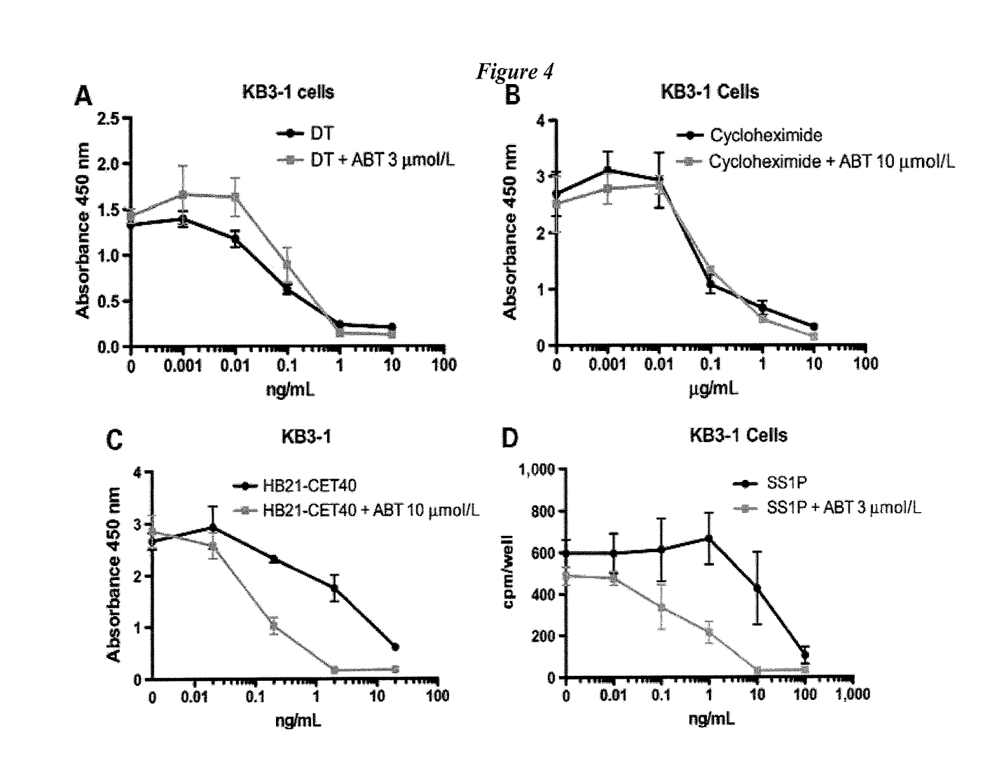

Given that the KB3-1 cells exhibited no apparent resistance to immunotoxin treatment, the 20-fold enhancement of HB21-PE40 and SS1P cytotoxicity by ABT-737 was surprising. To explore the mechanism of the ABT-737 effect, we did additional experiments using the same WST-1 in which ABT-737 was added in combination with three other agents that inhibit protein synthesis. These included native DT, cycloheximide, and HB21-CET40. HB21-CET40 is a newly described immunotoxin made from a truncated exotoxin derived from Vibrio cholerae, and ends in a KDEL (SEQ ID NO:4) like sequence (Sarnovsky R, et al., Cancer Immunol Immunother 2010; 59:737-46). Only ABT-737 in combination with PE- or cholera exotoxin (CET)-immunotoxins produced a >10-fold enhancement of toxicity (FIGS. 3A-C and 4C). These agents end in KDEL (SEQ ID NO:4) like sequences and are reported to translocate to the cytosol from the ER (Chaudhary V K, J et al., Proc Natl Acad Sci USA 1990; 87:308-12 and Jackson M E, et al., J Cell Sci 1999; 112:467-75). There was no evidence of ABT-737-mediated enhancement for DT and cycloheximide, which reach the cytosol by ER-independent routes, (FIGS. 4A and B). This result does not contradict the observation reported in FIG. 2C, in which there was an increase in caspase 3 activity with ABT-737 and cycloheximide, but rather reflects what each assay measures. The enhancement of PE cytotoxic activity could be due to the increased delivery of toxin or increased susceptibility of ABT-treated cells for ADP-ribosylated EF2. The lack of enhancement of DT cytotoxicity seemed to rule out the latter.

The effect of ABT-737 on the delivery of the enzymatic domain of PE to the cytosol was examined. If PE were delivered in greater amounts, one would expect a greater reduction in protein synthesis. Alternatively, if ABT-737 were acting downstream of ADP-ribosylation, then protein synthesis levels would be the same regardless of the presence of ABT-737. The findings indicated that for two PE-based immunotoxins (SS1 is shown), there was a 25-fold greater reduction in the level of protein synthesis in the presence of ABT (FIG. 4D, and data with HB21-PE40; data not shown) than in its absence. The results accord with ABT-737 promotion of the delivery of a greater number of PE molecules from the ER to the cytosol.

D. ABT-737 Treatment Produces Stress within the ER

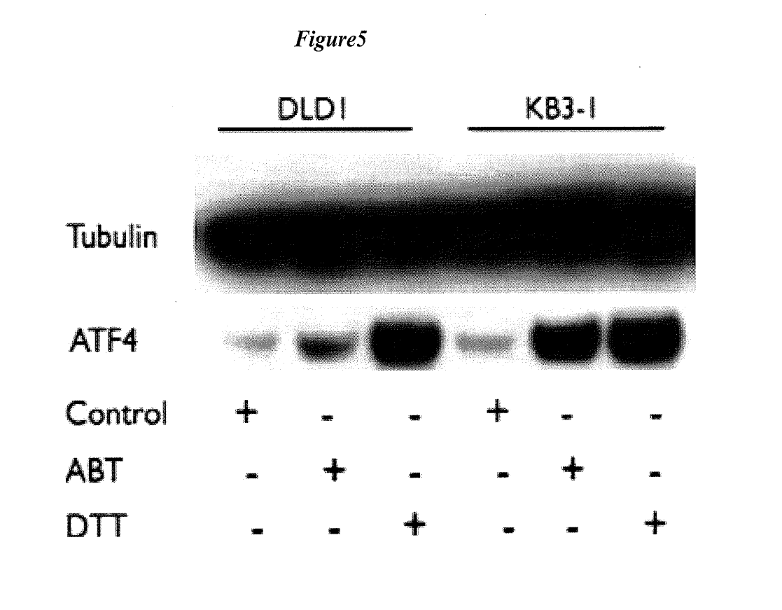

Immunoblot analysis is inadequate to detect the cellular uptake of immunotoxins, added at subnanomolar concentrations. Thus, we could not document directly that additional molecules of toxin were delivered to the cytosol. Instead, we sought other evidence that ABT-737 was directly or indirectly interacting with the ER. In various cell types, Bcl-2 family members are found associated with the ER (Oakes S A, et al., Proc Natl Acad Sci USA 2005; 102:105-10 and Scorrano L, et al., Science 2003; 300:135-9). To test if ABT-737 interacted with the ER and provoked an ER stress response, we incubated KB3-1 or DLD1 cells for 4 hours with either ABT-737 or with DTT; the latter being a well-known mediator of ER stress. Treated cell extracts were analyzed for the transcription factor ATF4 (Bernales S, Papa F R, Walter P. Annu Rev Cell Dev Biol 2006; 22:487-508). DTT strongly upregulated the ER stress response in both cell lines, whereas ABT-737 produced a strong stress response in KB3-1 cells and a moderate one in DLD1 cells (FIG. 5A). Overall, our results are consistent with ABT-737 acting on the ER and causing, either directly or indirectly, an increased translocation of PE- or CET-based immunotoxins to the cytosol.

### E. Dosage Considerations for Immunotoxin-ABT Combination Therapy

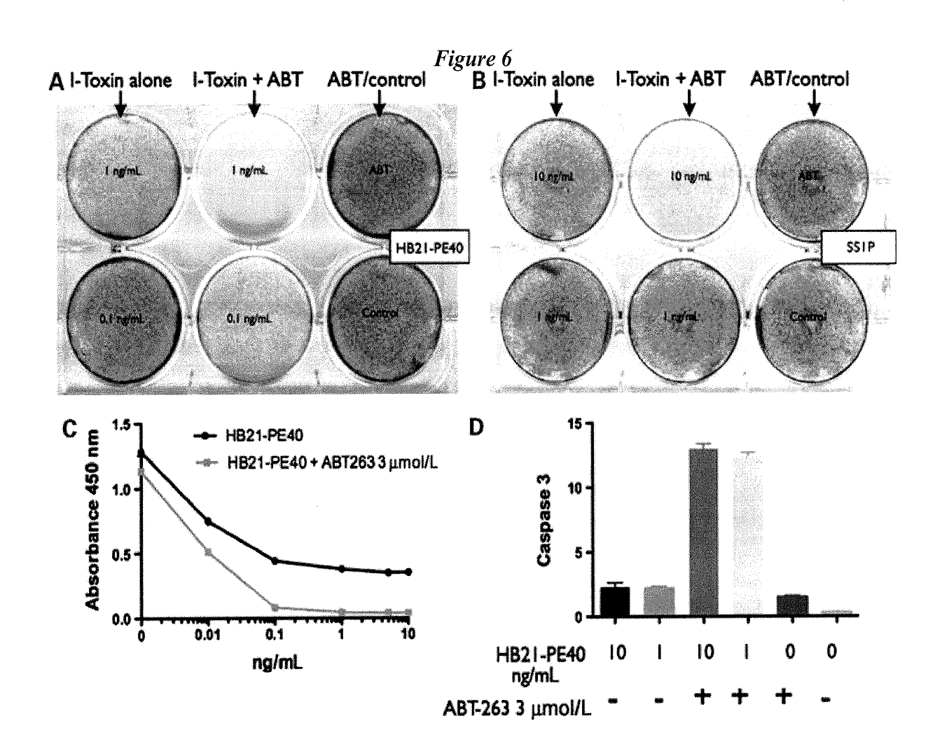

Assays for cell viability routinely involve exposing cells to toxins continuously for 48 hours. However, the plasma half-lives of PE-based recombinant immunotoxins are different and on the order of only 2 to 7 hours. To simulate short-term in vivo exposures, immunotoxin-ABT-737 combinations were applied in culture for 4 hours. The surviving cells were then evaluated after 6 days (FIGS. 6A and B). The immunotoxin HB21-PE40 in combination with ABT-737 provided a >10-fold enhancement of killing (FIG. 6A). Similarly, ABT-737 enhanced SS1P activity (FIG. 6B). In a parallel experiment, a 4-hour exposure with cycloheximide and ABT-737 did not result in any significant cell killing (data not shown). The subject immunotoxins act catalytically and have no known intracellular inhibitors and accordingly should continue to be active once delivered to cytosol. However, given that cycloheximide is a reversible inhibitor of protein synthesis, the effect of cycloheximide would only be noted if the compound were present continually. Accordingly, ABT-737 should be particularly useful in promoting immunotoxin killing even with short-term exposures.

An “orally available” variant of ABT-737, ABT-263, is being clinically tested. To show that ABT-263 behaves similarly as ABT-737, additional cytotoxicity and caspase 3 activity assays were conducted with it (FIGS. 6C and D). DLD1 cells were incubated with HB21-PE40 in the presence or absence of ABT-263. ABT-263 showed similar toxicity enhancing activities as seen for ABT-737 (FIGS. 6C and D). ABT-263 also similarly enhanced KB3-1 and SKOV3 killing (data not shown). Accordingly, ABT-263 can overcome immunotoxin resistance.

In summary, we found that complete inhibition of protein synthesis and loss of Mcl-1 does not always lead to cell death. To overcome this resistance, we used the BH3-only mimetic, ABT-737, which implicates Bcl-2, Bcl-xl, or Bcl-w as the proximate cause of resistance. In addition, we unexpectedly discovered that ABT-737 enhances the delivery of active PE to the cysotol suggesting that prosurvival Bcl-2 proteins are located in the ER and provided data indicating that ABT-737 disrupts ER function via the neutralization of one or more of these proteins. Our data support the preclinical development of ABT-263-immunotoxin combinations for cancer therapy and the use of ABT-263 to enhance the delivery to the cytosol of other agents targeted to the endoplasmic reticulum.

### Example 2

### Synergy Between ABT-737 Treatment and a PE38 or PE40 in KB Adenocarcinoma Cells

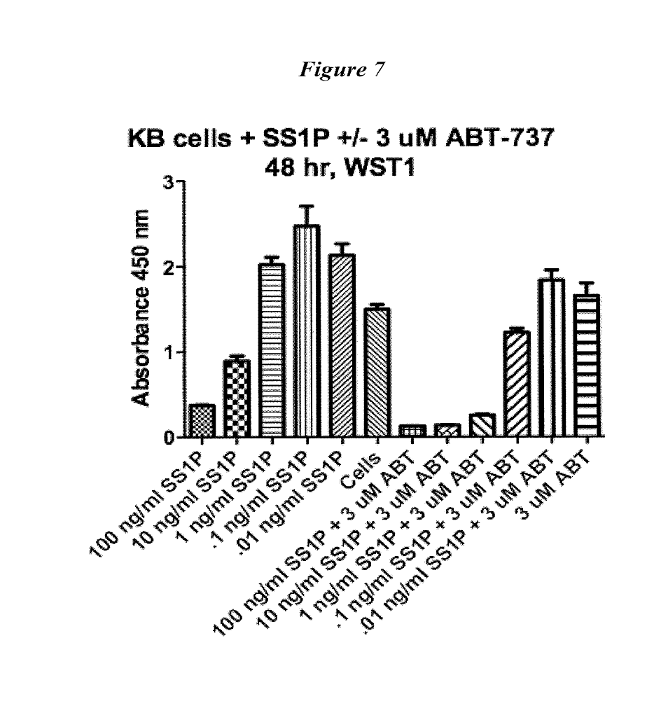

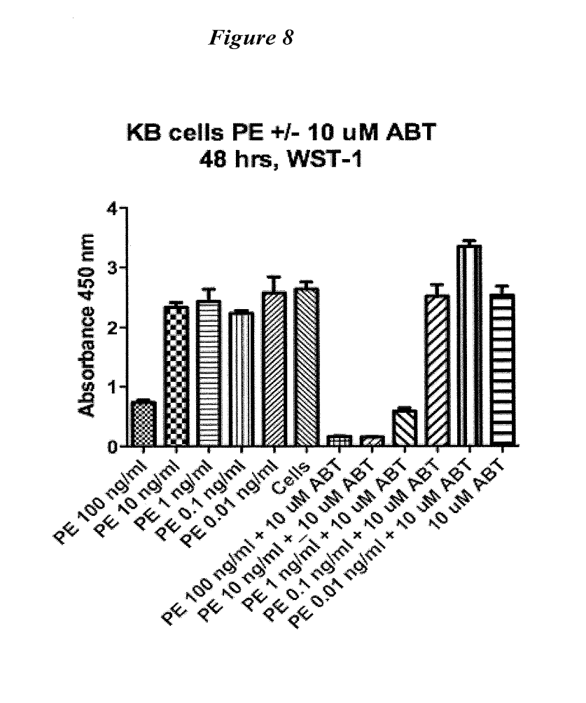

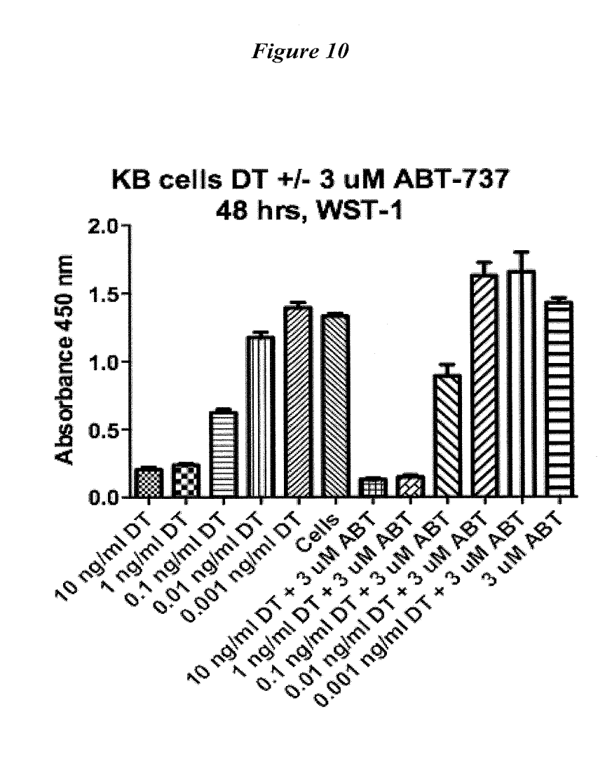

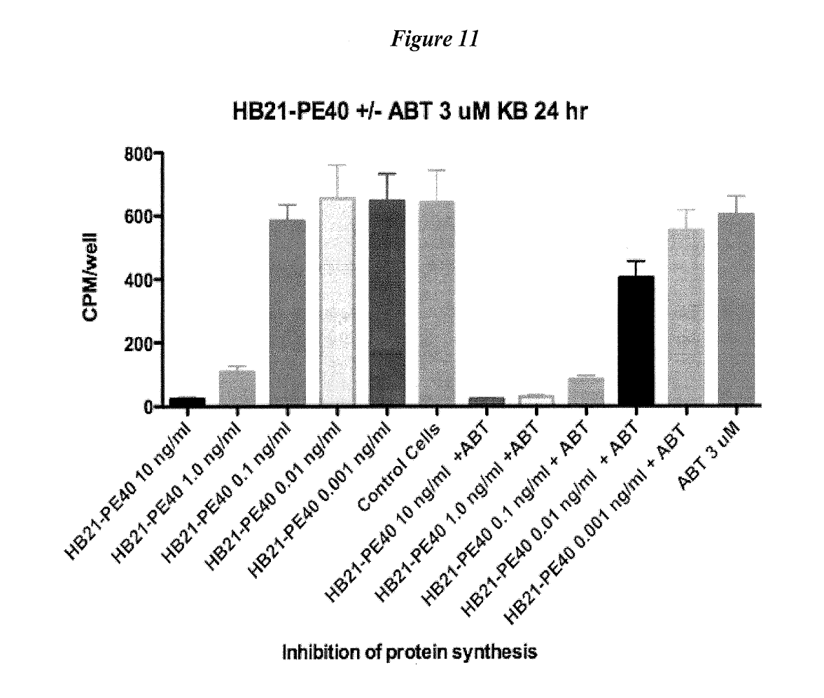

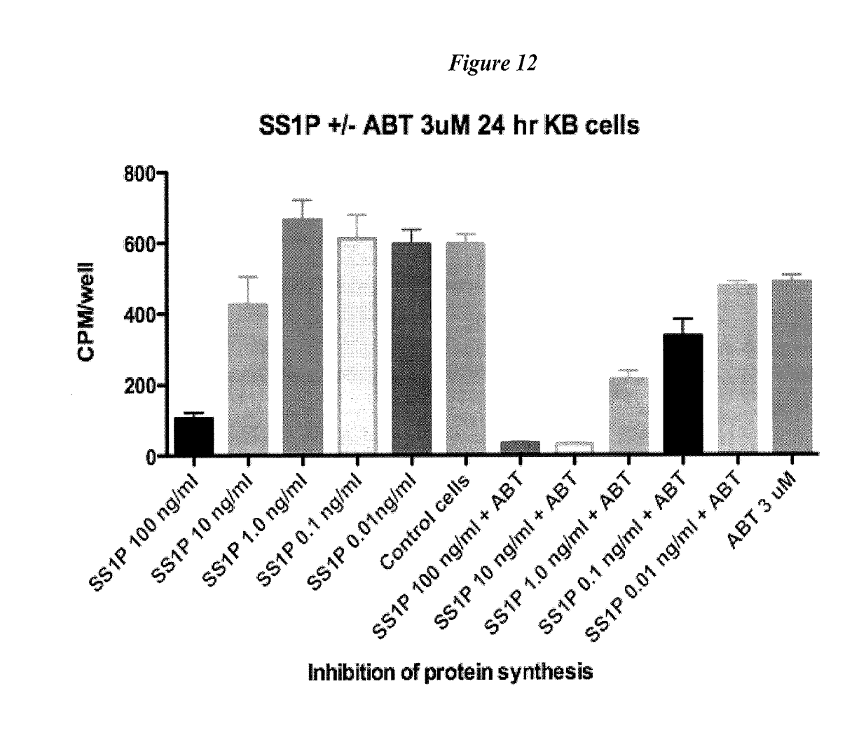

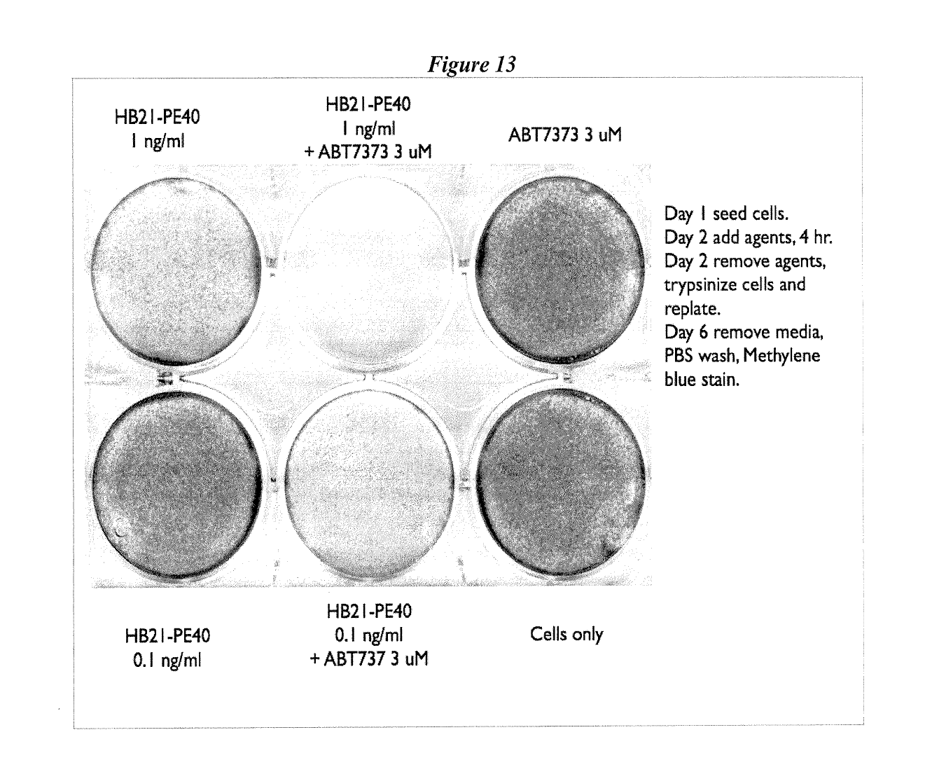

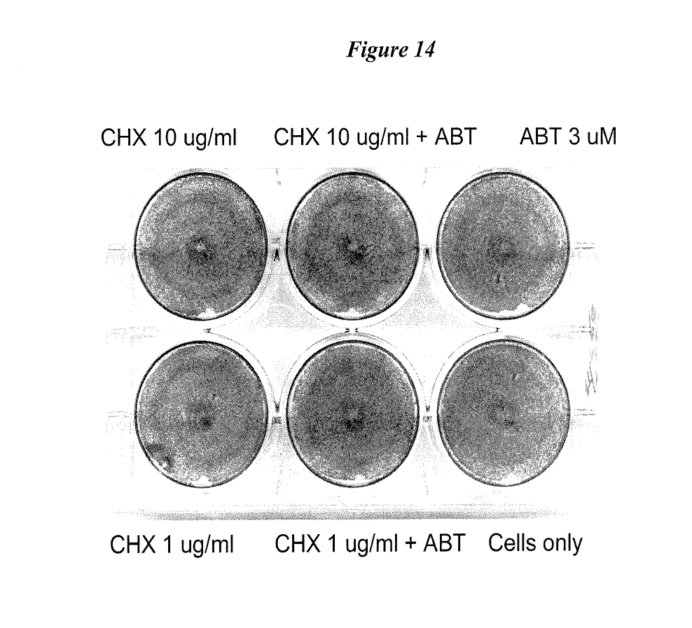

FIGS. 7 to 14 illustrate the highly synergistic pro-apoptotic interaction between APT-737 treatment and PE-immunotoxin treatment as compared to the much weaker interaction observed for APT-737 treatment and cycloheximide and diptheria toxins.

### Example 3

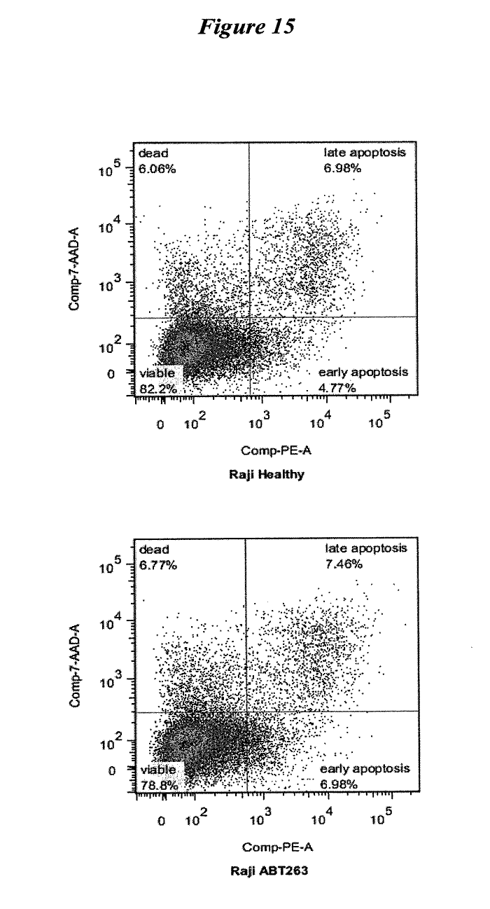

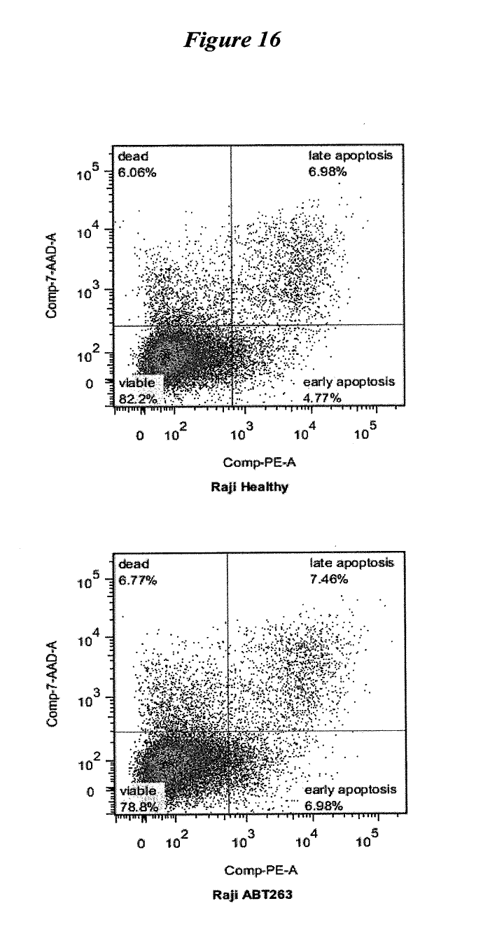

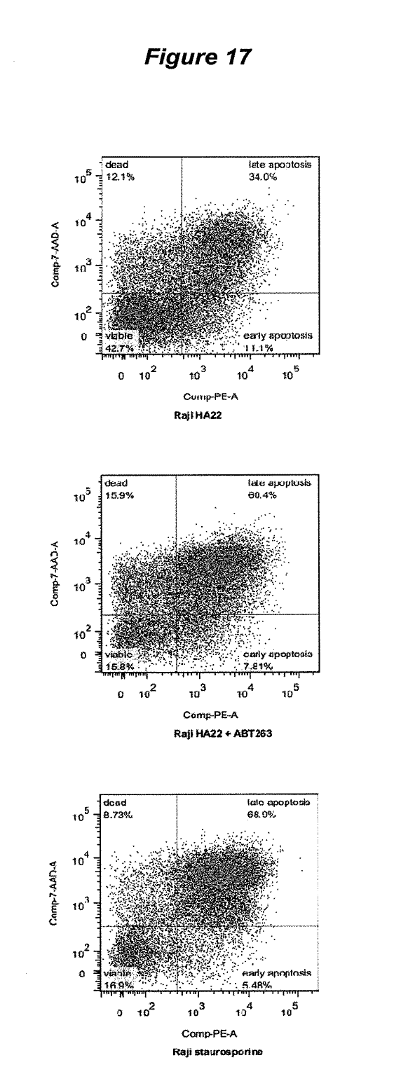

BL22 and HA22 are variants of the same basic immunotoxin targeted to CD22 on B-cell malignancies. The effects of these agents on Raji cells are shown in FIGS. 15 to 17. The intensity of staining for Annexin V is shown on the X-axis. Annexin V binds to phosphatidylserine. Dying cells that undergo apoptosis display phosphatidylserine, on their cell surface. Phosphatidylserine is normally found on the cytosolic surface of the plasma membrane, but is redistributed during apoptosis to the extracellular surface. The y axis shows staining with a dye 7-AAD that is normally excluded from living cells. When cells die, they take up the dye. Cells that are positive only for Annexin staining are deemed “early” apoptosis. Cells that are positive for Annexin and 7-AAD are in “late” apoptosis. Cells that are positive for 7-AAD but negative for Annexin are considered dead—but with an unknown mechanism. FIG. 15 shows that little or no apoptosis is mediated by ABT-263 alone. FIG. 16 shows that the low affinity immunotoxin (low affinity for CD22) causes 47% total apoptosis in 24 hrs. However, when ABT-263 was added in combination, this amount of apoptosis increased to 62%—also in 24 hrs. FIG. 17 shows the results for the high affinity immunotoxin (high for CD22)-45% total apoptosis. Apoptosis increased to 68% when ABT was present. The last panel of FIG. 17 is a positive control for generating “maximal” apoptosis in 24 hrs—and shows ˜74% apoptosis.

### Example 4

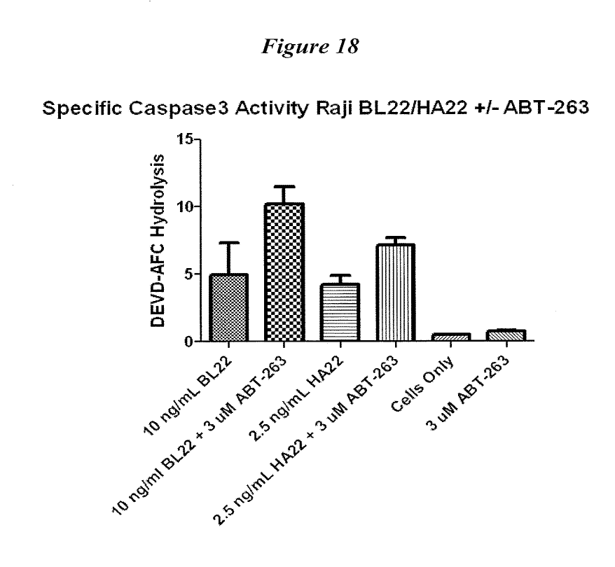

10⁶ RAji cells were plated per well, 6 well plate, 2.5 mL total. They were immediately treated with BL22, HA22, ABT 263 formulated in DMSO (Toronto Research Chemicals Inc) and incubated for 24 hours. The initiation of apoptosis can be measured in several ways. Quantification is often performed with a caspase enzyme assay. This enzyme assay measures the activation of caspase 3, the final enzyme in the apoptosis cascade. Their caspase 3 levels were then determined using the R&D Caspase-3 Fluorometric Assay in which the cells are lyszed in the provided lysis buffer, incubated for two hours with reaction mixture containing: reaction buffer, DEVD-AFC (SEQ ID NO:9) substrate, and DTT; and the fluorescence signal detected with a fluorimeter (Cary Eclipse) at 505 nm after 2 hours of incubation with kit. The results are shown in FIG. 18. The caspase 3 results support the flow cytometry result. Untreated cells have a low background level for this enzyme. Immunotoxin treatment alone, produces less than optimal caspase activation, while the combination produces the most.

### Example 5

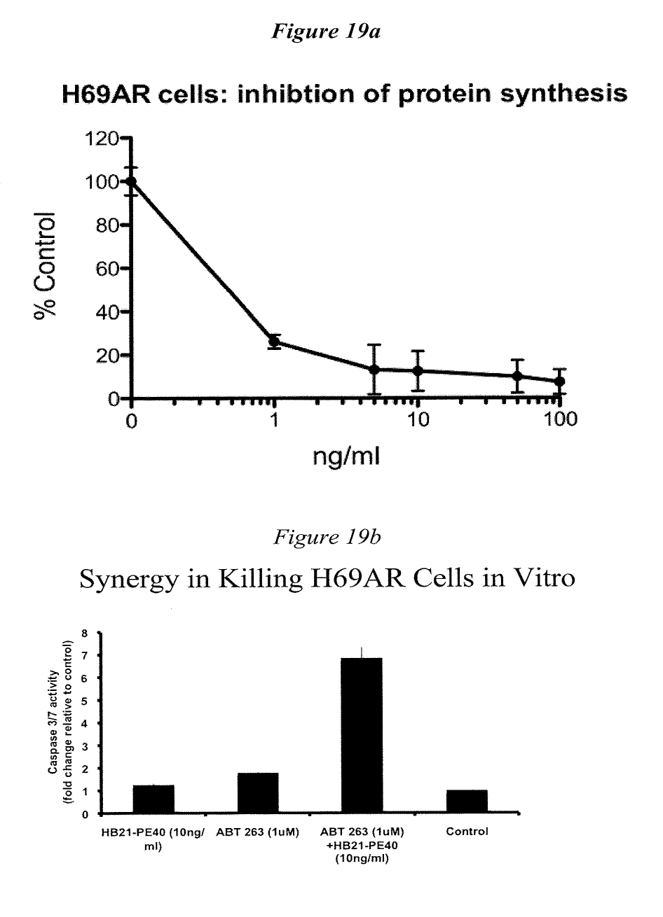

Small cell lung cancers are variably sensitive to ABT-263/737. H69AR cells are resistant to ABT-263/737. They are also resistant to killing by the immunotoxin HB21-PE40, despite inhibition of protein synthesis (See, FIG. 19a). In tissue culture, the combination of HB21-PE40 and ABT-263/737 is synergistic and leads to cell death via apoptosis (See, FIG. 19b).

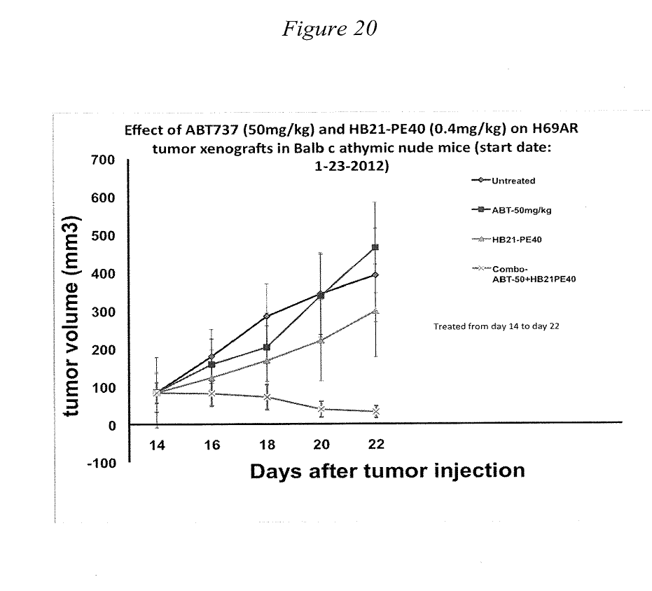

To determine if the combination is synergistic in vivo, tumor cells H69AR were injected SC on flank of Balb/c nude mice. After 14 days, ABT-737 was injected IP at 50 mg/kg and HB21-PE40 at 0.4 mg/kg also IP. Treatment was given daily for 8 days. The results are shown in FIG. 20. Combination treatment of SC implanted H69AR tumor cells evidenced a much greater than additive killing of the resulting tumors than either treatment separately.

### Example 6

This example investigates combination treatment with ABT-737 and SS1P on inducing cell death in pancreatic cancer cell lines. Pancreatic cancer cell line KLM1 is very sensitive to SS1P+ABT737. Here, other pancreatic cancer cell lines are also investigated, including those with minimal mesothelin expression, for their response to SS1P+ABT737.

Methods: All 4 lines were treated for 24 hr with SS1P (300 ng/ml), ABT737 (10 uM) and SS1P+ABT737. The following was done for each individual pancreatic cancer cell line: 4×10⁵ cells (in a 2 ml suspension of RPMI medium) per well were seeded in 4 wells of a 6-well plate. After 24 hrs in the incubator (37° C. and 5% CO2), all medium was removed. One well was treated with 2 ml of SS1P (300 ng/ml), one with 2 ml of ABT-737 (10 uM), and one with 2 ml SS1P (300 ng/ml)+ABT-737 (10 uM). In the remaining well, cells were not treated and 2 ml of fresh RPMI medium was added. Dilutions of SS1P and ABT-737 were made in RPMI medium. Cells were not pretreated, and both components of the combination treatment were given simultaneously

Cells were treated for a period of 24 hrs in the incubator (37° C. and 5% CO2). After that, both floating and adherent cells were collected for FACS analysis. Adherent cells were collected with 0.05% Trypsin-EDTA (Invitrogen).

FACS analysis was done with the PE Annexin V Detection Kit I (BD):

1. Wash cells twice with cold PBS and then resuspend cells in 1× Binding Buffer at a concentration of 1×10̂6 cells/ml.
2. Transfer 100 μl of the solution (1×10̂5 cells) to a 5 ml culture tube.

### 3. Add 5 μl of PE Annexin V and 5 μl 7-AAD.

4. Gently vortex the cells and incubate for 15 min at RT (25° C.) in the dark.
5. Add 4000 of 1× Binding Buffer to each tube. Analyze by flow cytometry within 1 hr.

FACS was performed on a FACS Calibur at the NCI FACS facility in Bldg 37. The obtained FACS data were analyzed with the software FlowJo. FACS data of the untreated cells was used to delineate the viable cells (7-AAD negative and PE Annexin V negative) with a quadrant gate (Q1, Q2, Q3 and Q4). The established gate was subsequently applied to the FACS data of the treated cells (=SS1P, ABT737, SS1P+ABT737), which allowed to identify the treatment effects as compared to the untreated cells. Q4 (7-AAD negative and PE Annexin V negative) are the % of viable cells, Q3 (7-AAD negative and PE Annexin V positive) are the % of cells in early apoptosis, Q2 (7-AAD positive and PE Annexin V positive) are the % in end stage apoptosis, and Q1 (7-AAD positive and PE Annexin V negative cells) are the % of in a further advanced stage of death. The number of death cells is consequently obtained by subtracting 100% with the % of viable cells (=Q4).

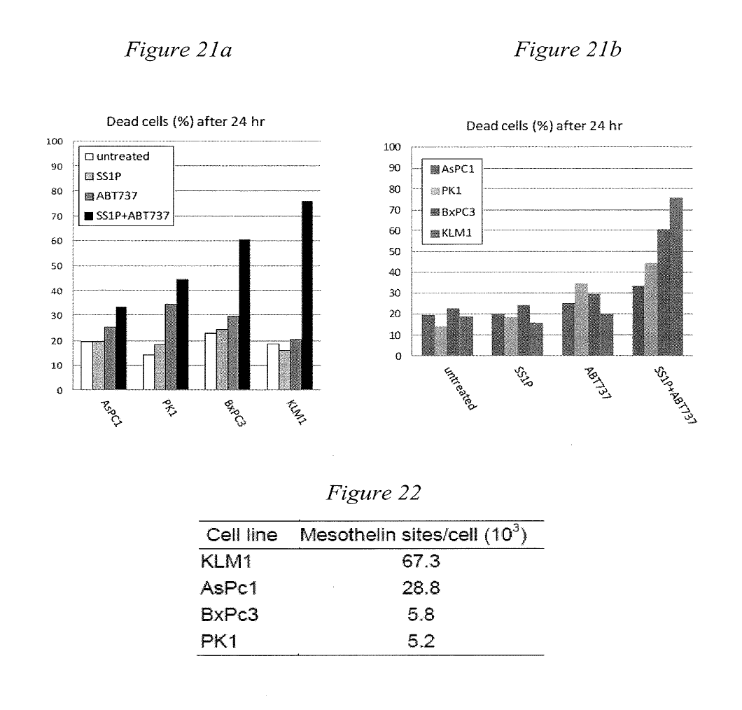

The results of the experiment are shown in FIGS. 21a and b. The FACS results themselves are not shown. As compared to the other cell lines, KLM1 is most affected by SS1P+ABT737, and also has the highest # of mesothelin binding sites per cell (See, FIG. 22). However, the response to SS1P+ABT737 does not seem to correlate with the number of mesothelin binding sites per cell. In contrary, AsPC1 is least sensitive to SS1P+ABT737, although it has a substantially higher number of mesothelin binding sites than BxPC3 and PK1. Interestingly, the latter two cell lines show a higher response to ABT737 alone. Of note, the untreated cells show up to 20% death, likely because of lengthy procedure—a result of the high # of samples that needed to be processed.

Surprisingly, even cell lines with a minimal mesothelin expression are sensitive to SS1P+ABT737. The factors which determine SS1P+ABT737 sensitivity are consequently not limited to mesothelin expression. Cell lines might differ in native expression levels of the apoptotic proteins, for example, and also in the effects treatment has on their protein levels.

The referenced patents, patent applications, and scientific literature, including accession numbers to GenBank database sequences, referred to herein are hereby incorporated by reference in their entirety as if each individual publication, patent or patent application were specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter. This incorporated material is also specifically incorporated with reference to the BH3-mimetic agents disclosed they disclose. This application claims priority benefit of U.S. Provisional Patent Application Ser. No. 61/238,032 which is incorporated herein by reference in its entirety including the figures, methods of treatment, and composition subject matter disclosed therein.

As can be appreciated from the disclosure above, the present invention has a wide variety of applications. The invention is further illustrated by the following examples, which are only illustrative and are not intended to limit the definition and scope of the invention in any way.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A composition comprising a chimeric molecule and a pro-apoptotic agent, wherein the chimeric molecule comprises a toxin that inhibits protein synthesis, wherein the pro-apoptotic agent inactivates a Bcl-2 family protein.

2. The composition of claim 1, wherein the chimeric molecule is an immunotoxin comprising an antibody against a cell surface antigen on a tumor cell and a toxin that inhibits protein synthesis.

3. (canceled)

4. The compositions of claim 2, wherein the cell surface antigen is selected from the group consisting of CD19, CD21, CD22, CD25, CD30, CD33, CD79b, transferrin receptor, EGF receptor, mesothelin, cadherin and Lewis Y.

5. (canceled)

6. The composition of claim 1, wherein the toxin is a Pseudomonas exotoxin A.

7. The composition of claim 6, wherein the Pseudomonas exotoxin A is selected from the group consisting of PE25, PE35, PE38, PE40, Domain III of PE, and variants thereof.

8. The composition of claim 1, wherein the pro-apoptotic agent is a BH3-only mimetic.

9. The composition of claim 1, wherein the pro-apoptotic agent selected from the group consisting of ABT-737, ABT-263, oblimersen sodium, AT-101 and GX15-070.

10. The composition of claim 2, wherein the antibody is selected from the group consisting of B3, RFB4, SS1, SS1P, SS1P-LR, MN and HB21.

11. The composition of claim 2, wherein the immunotoxin is selected from the group consisting of LMB-2, LMB-7, LMB-9, BL22, HA22, HA22-LR, SS IP and SS1P-LR.

12. The composition of claim 2, wherein the pro-apoptotic agent is encapsulated in a liposome that is attached to the immunotoxin.

13. A method of killing a tumor cell comprising contacting the cell with a chimeric molecule and a pro-apoptotic agent, wherein the chimeric molecule comprises a toxin that inhibits protein synthesis, wherein the pro-apoptotic agent inactivates a Bcl-2 family protein.

14. The method of claim 13, wherein the chimeric molecule is an immunotoxin comprising an antibody against a cell surface antigen on a tumor cell and a toxin that inhibits protein synthesis.

15. The method of claim 14, wherein the cell surface antigen is on a lymphocytic cell.

16. The method of claim 14, wherein the cell surface antigen is selected from the group consisting of CD19, CD21, CD22, CD25, CD30, CD33, CD79b, transferrin receptor, EGF receptor, mesothelin, cadherin and Lewis Y.

17. (canceled)

18. (canceled)

19. The method of claim 13, wherein the toxin is selected from Pseudomonas exotoxin A, diphtheria toxin, cholix toxin, cholera exotoxin, shiga toxin, ricin toxin and pokeweed antiviral protein (PAP).

20. The method of claim 13, wherein the toxin is a Pseudomonas exotoxin A.

21. The method of claim 20, wherein the Pseudomonas exotoxin A is selected from the group consisting of PE25, PE35, PE38, PE40, Domain III of PE, and variants thereof.

22. The method of claim 13, wherein the pro-apoptotic agent is a BH3-only mimetic.

23. The method of claim 13, wherein the pro-apoptotic agent selected from the group consisting of ABT-737, ABT-263, oblimersen sodium, AT-101 and GX15-070.

24. The method of claim 14, wherein the antibody is selected from the group consisting of B3, RFB4, SS1, MN and HB21.

25. The method of claim 14, wherein the immunotoxin is selected from the group consisting of LMB-2, LMB-7, LMB-9, BL22, HA22, HA22-LR, SS1P and SS1P-LR.

26. The method of claim 14, wherein the pro-apoptotic agent is encapsulated in a liposome that is attached to the immunotoxin.

27. The method of claim 14, wherein the immunotoxin and the pro-apoptotic agent are administered together.

28. The method of claim 14, wherein the immunotoxin and the pro-apoptotic agent are administered separately.

29. (canceled)

30. The method of claim 29, wherein the toxin comprises an endoplasmic reticulum retention sequence.

31-32. (canceled)
